(19) United States
(12) Patent Application Publication
(10) Pub. No.: US 2016/0235842 A1
(43) Pub. Date: Aug. 18, 2016

(54) MEDICAL USES OF CD38 AGONISTS

(71) Applicant: THE BOARD OF TRUSTEES OF THE LELAND STANFORD JUNIOR UNIVERSITY, Palo Alto, CA (US)

(72) Inventors: Matthew J. Goldstein, Hillsborough, CA (US); Ronald Levy, Stanford, CA (US); Holbrook Kohrt, San Francisco, CA (US)

(73) Assignee: The Board of Trustees of the Leland Stanford Junior University, Stanford, CA (US)

(21) Appl. No.: 14/904,814

(22) PCT Filed: Jul. 15, 2014
(86) PCT No.: PCT/US2014/046705

§ 371 Date: Jan. 13, 2016

Related U.S. Application Data
(60) Provisional application No. 61/846,372, filed on Jul. 15, 2013.

(51) Int. Cl. A61K 39/395; C07K 16/32; G01N 33/574; C07K 16/28

(52) U.S. Cl. CPC A61K 39/3955; A61K 39/39558; C07K 16/2896; C07K 16/2878; C07K 16/2887; C07K 16/32; G01N 33/57492; A61K 2039/507

(57) ABSTRACT

Methods and compositions relating to medical (e.g., therapeutic) use of CD38 agonists are provided. In some embodiments, the present invention provides methods and compositions relating to use of CD38 in the treatment of cancer, particularly to enhance the efficacy of antibody therapy directed to cancer cells.

### GOVERNMENT RIGHTS

This invention was made with Government support under contract CA153248 awarded by the National Institutes of Health. The Government has certain rights in this invention.

### BACKGROUND

Monoclonal antibody therapy is rapidly becoming the standard of care for many diseases, disorders, and conditions, including certain cancers. Despite the promising activity of monoclonal antibodies, the response rates among patients with either refractory or advanced cancer often are only partial, less than 25%, due to various factors.

### SUMMARY

The present invention demonstrates effective treatment of cancer by agonizing CD38 (i.e., by administration of CD38 agonist therapy, for example comprising administering a CD38 agonist). Prior to the present invention, CD38 expressed by tumor cells had been described as a potential target for inhibitory therapy to treat cancer. According to prior understanding, therefore, agonizing CD38 would be affirmatively undesirable for cancer patients. The present invention, however, establishes that administration of a CD38 agonist can enhance the ability of a subject's immune system to target and destroy cancer cells. In particular, the present invention demonstrates that agonizing CD38 can increase effector cell killing of tumor cells. As is known in the art, one of the mechanisms by which immune effector cells (e.g., natural killer [NK] cells, macrophages, neutrophils, eosinophils) exert their effects is by destroying target cells that have been “labeled” (i.e., bound) by antibodies (e.g., that interact with a marker on the target cell surface). This process is known as antibody-dependent cellular cytotoxicity (ADCC). The present disclosure specifically demonstrates that administration of a CD38 agonist can augment the ADCC capability of immune effector cells such as NK cells, in particular against cancer cells to which an antibody has bound.

The present invention further demonstrates the utility and effectiveness of combining CD38 agonist therapy with anti-tumor antibody therapy. Still further, the present invention demonstrates the utility and effectiveness of a serial, staged CD38 agonist therapy relative to anti-tumor antibody therapy. Specifically, the present invention explicitly establishes that CD38 levels on immune effector cells can be enhanced by contact with tumor cells bound by anti-tumor antibody (i.e., antibody that specifically binds to a tumor antigen). Still further, the present invention demonstrates that administration of a CD38 agonist after such enhancement achieves remarkably effective killing of the tumor cells. The effectiveness of such combination therapy, and particularly of such staged combination therapy, is particularly surprising in light of CD38's significant expression on cells other than immune effector cells, and particularly in light of CD38's expression on cancer cells.

In one aspect, the present invention provides methods of treating cancer by administering to a patient a composition comprising a CD38 agonist. In some embodiments, such administration is to a subject (e.g., a patient) who is receiving or has received anti-tumor antibody therapy. In some embodiments, the subject has received anti-tumor antibody therapy a period of time prior to the administering of the CD38 agonist. In particular such embodiments, the period of time is selected so that, prior to the administering of the CD38 agonist, CD38 expression has increased on surfaces of effector cells that mediate antibody-dependent cellular cytotoxicity (ADCC) upon exposure to tumor cells bound by an anti-tumor antibody. In many embodiments, and particularly in many embodiments in which CD38 agonist therapy is combined with anti-tumor antibody therapy (especially when administration of the CD38 agonist therapy is delayed a period of time relative to administration of the anti-tumor antibody therapy) administration of such therapy results in increased ADCC, presumably mediated by such effector cells with increased CD38 surface expression increase their ADCC. In some embodiments, apoptosis of the tumor cells upon administering of the CD38 agonist is increased relative to that observed absent the CD38 agonist. In some embodiments, tumor growth upon administering of the CD38 agonist is reduced relative to that observed absent the CD38 agonist.

In some embodiments, the present invention provides methods of administering CD38 agonist therapy together with agonist therapy directed at one or more inducible immune effector cell surface markers other than CD38. In some embodiments, such methods further involve administering anti-tumor antibody therapy. Thus, in some embodiments, the present invention provides methods of treating cancer that include steps of i) administering anti-tumor antibody therapy; ii) administering anti-CD38 agonist therapy; and iii) administering agonist therapy targeting at least one inducible immune effector cell surface marker other than CD38. In some embodiments, (at least one dose of) CD38 agonist therapy is administered a first period of time after (at least one particular dose of) anti-tumor antibody therapy. In some embodiments, agonist therapy targeting at least one inducible immune effector cell surface marker other than CD38 is administered a second period of time after (at least one particular dose of, optionally the same particular dose of) anti-tumor antibody therapy. In some embodiments, the first and second time periods are relative to the same dose of anti-tumor antibody therapy. In some embodiments, the first and second time periods are the same. In some embodiments, the first and second time periods are different.

In some embodiments, methods of the present invention involve determining a CD38 expression level on effector cells in a subject. In some such embodiments, CD38 expression level is determined prior to administering a CD38 agonist. In some embodiments, CD38 expression level is determined at multiple time points. In some embodiments, CD38 expression level is determined before, substantially simultaneously with, and/or after administration of one or more doses of a CD38 agonist. In some embodiments, CD38 expression level is determined before, substantially simultaneously with, and/or after administration of one or more doses of an anti-tumor antibody therapy. In some embodiments, CD38 expression level is determined after administration of (at least a particular dose of) anti-tumor antibody therapy and before administration of (at least a particular dose of) CD38 agonist therapy. In some embodiments, (at least one dose of) anti-tumor antibody therapy is administered, followed by a delay that lasts a period of time, and then (at least one dose of) CD38 agonist therapy is delivered, and CD38 expression level is determined at least once, and optionally multiple times, within the period of time and/or prior to the anti-tumor antibody therapy; in some such embodiments, the period of time has a length determined by a change in determined CD38 expression level. That is, in some embodiments, CD38 agonist therapy is not administered until an increase (e.g., a significant increase) in CD38 level on immune effector cells is determined after administration of the anti-tumor antibody therapy.

In some embodiments, methods of the present invention involve determining an expression level of an inducible immune effector cell surface marker other than CD38, on immune effector cells in a subject. In some such embodiments, such expression level is determined prior to administering an agonist of the relevant inducible immune effector cell surface marker. In some embodiments, such an expression level is determined at multiple time points. In some embodiments, such an expression level is determined before, substantially simultaneously with, and/or after administration of one or more doses of agonist therapy targeting the an inducible immune effector cell surface marker other than CD38. In some embodiments, such expression level is determined before, substantially simultaneously with, and/or after administration of one or more doses of an anti-tumor antibody therapy. In some embodiments, expression level of the an inducible immune effector cell surface marker other than CD38 is determined after administration of (at least a particular dose of) anti-tumor antibody therapy and before administration of (at least a particular dose of) agonist therapy targeting the an inducible immune effector cell surface marker other than CD38. In some embodiments, (at least one dose of) anti-tumor antibody therapy is administered, followed by a delay that lasts a period of time, and then (at least one dose of) agonist therapy is delivered, and expression level is determined at least once, and optionally multiple times, within the period of time and/or prior to the anti-tumor antibody therapy; in some such embodiments, the period of time has a length determined by a change in determined expression level of the an inducible immune effector cell surface marker other than CD38. That is, in some embodiments, agonist therapy is not administered until an increase (e.g., a significant increase) in expression level an inducible immune effector cell surface marker other than CD38 on immune effector cells is determined after administration of the anti-tumor antibody therapy.

In some embodiments of methods of the present invention, expression levels both of CD38 and of an inducible immune effector cell surface marker other than CD38 are determined. In some embodiments, both such expression levels are determined at the same time. In some embodiments, such expression levels are determined at different times. In some embodiments, expression levels of CD38 and/or of an inducible immune effector cell surface marker other than CD38 may be determined at multiple times, some or all of which may be, but need not be, at the same time.

In some embodiments, CD38 expression level, and/or expression level of an inducible immune effector cell surface marker other than CD38, on immune effector cells is determined in a patient sample (e.g., a primary sample or a secondary sample derived by processing a primary sample). In some such embodiments, the patient sample is or comprises a blood sample. In some embodiments, the patient sample is or comprises a tissue sample. In some embodiments, the patient sample is or comprises a tumor sample (e.g., includes tumor cells). In some embodiments, an inducible immune effector cell surface marker other than CD38 is selected from a group consisting of a member of the TNFR family, a member of the CD28 family, a cell adhesion molecule, a vascular adhesion molecule, a G protein regulator, an immune cell activating protein, a recruiting chemokine/cytokine, a receptor for a recruiting chemokine/cytokine, an ectoenzyme, a member of the immunoglobulin superfamily, a lysosomal associated membrane protein.

Thus, among other things, the present invention provides improved methods of treating cancer with anti-tumor antibody therapy, which improvement comprises combining the anti-tumor antibody therapy with CD38 agonist therapy as described herein. In some embodiments, the improvement further comprises administering CD38 agonist therapy a period of time after administration of (at least a one particular dose of) anti-tumor antibody therapy. In some embodiments, the improvement further comprises combining the anti-tumor antibody therapy with agonist therapy targeting an inducible immune effector cell surface marker other than CD38, in addition to the CD38 agonist therapy. In some such embodiments, the improvement further comprises administering agonist therapy targeting the inducible immune effector cell surface marker other than CD38 a period of time after administration of (at least one particular dose of) anti-tumor antibody therapy. In some embodiments, the improvement is reflected in increased ADCC (e.g., mediated by immune effector cells expressing CD38).

Furthermore, among other things, the present invention provides methods of enhancing antibody-dependent cellular cytotoxicity (ADCC) of effector cells (e.g., NK cells) in a subject, which methods involve administering to the subject a CD38 agonist therapy.

In some embodiments, CD38 agonist therapy comprises administration of one or more doses of a CD38 agonist according to a regime correlated with elevated ADCC of the effector cells (e.g., as compared with a level observed under otherwise comparable conditions lacking such administration). In some embodiments, ADCC is evaluated by a chromium release assay. In some particular embodiments in which the ADCC of effectors cells is increased, the degranulation of such effector cells is increased (e.g., relative to that observed under otherwise comparable conditions lacking the CD38 agonist). Alternatively or additionally, in some embodiments, mobilization of CD107a on the surface of such effector cells is increased (e.g., relative to that observed under otherwise comparable conditions lacking the CD38 agonist). In some embodiments, cytokine release from such effector cells is increased (e.g., relative to that observed under otherwise comparable conditions lacking the CD38 agonist).

In some embodiments, subjects to which methods provided by the present invention are applied or administered are suffering from cancer. In some embodiments, the cancer is selected from a group of hematologic malignances including acute lymphoblastic leukemia, acute myeloid leukemia, chronic lymphocytic leukemia, chronic myelogenous leukemia, hairy cell leukemia, AIDS-related lymphoma, Hodgkin lymphoma, non-Hodgkin lymphoma, Langerhans cell histiocytosis, multiple myeloma, and myeloproliferative neoplasms. In some embodiments, the cancer is selected from a group of solid tumors including breast carcinoma, squamous cell carcinoma, colon cancer, head and neck cancer, lung cancer, genitourinary cancer, rectal cancer, gastric cancer, and esophageal cancer.

In some embodiments, subjects to which methods provided by the present invention are applied or administered are receiving or have received at least one dose of anti-tumor antibody therapy. In some embodiments, such subjects have received a particular dose of anti-tumor antibody therapy a specific period of time prior to application or administration of one or more steps of provided methods. In particular, in some embodiments, subjects have received a particular dose of anti-tumor antibody therapy a specific period of time prior to receiving CD38 agonist therapy.

The present invention provides certain CD38 agonist agents and pharmaceutical compositions thereof. In some embodiments, a CD38 agonist is or comprises a non-antibody agent. In some embodiments, such a non-antibody agent is or comprises an aptamer that specifically binds to CD38. In some embodiments, a CD38 agonist is or comprises an antibody agent that specifically binds to CD38 (e.g., to CD38 on surfaces of immune effector cells). In some embodiments, such an antibody agent is or comprises an intact antibody. In some embodiments, such an antibody agent is or comprises a monoclonal antibody (mAb). In some embodiments, such an antibody agent is or comprises a humanized or human antibody, or includes antigen binding elements of a human or humanized antibody. In some embodiments, such an antibody agent is a multi-specific agent, such as a bi-specific antibody. In some such embodiments, the multi-specific agent binds specifically to CD38 and to an inducible immune effector cell surface marker other than CD38. In some embodiments, the multi-specific agent binds specifically to CD38 and to a tumor antigen. In some embodiments, the multi-specific agent binds specifically to CD38 and to another antigen, which other antigen is not a tumor antigen (so that CD38 and tumor antigen are not simultaneously targeted). In some embodiments that utilize a multi-specific agent that binds specifically to both CD38 and a tumor antigen, the multi-specific agent is not administered to a subject until a period of time has passed (e.g., sufficient to permit an increase in CD38 expression on surfaces of immune effector cells) after the subject received anti-tumor antibody therapy with an antibody agent that did not also target CD38.

Still further, the present invention provides a variety of kits or articles of manufacture containing components relevant to administration of CD38 agonist therapy and/or detection of CD38 expression, particularly on surfaces of immune effector cells, particularly in samples from patients (e.g., those who have received or are receiving anti-tumor antibody therapy and/or CD38 agonist therapy).

The present invention provides a method of treating cancer in a patient who has received anti-tumor antibody therapy, the method comprising: administering to the patient a composition comprising a CD38 agonist, the administering being performed a period of time after the anti-tumor antibody therapy, such that CD38 expression has increased on surfaces of effector cells that mediate antibody-dependent cellular cytotoxicity (ADCC) when such cells are exposed to tumor cells bound by an anti-tumor antibody; the CD38 agonist being characterized in that, when the effector cells with increased CD38 expression on their surface are contacted with the agonist, their ADCC is increased as compared with that observed absent such contact.

In some embodiments, the method of treating cancer further comprises at least one step of determining CD38 expression level on surfaces of the effector cells, the determining being performed prior to the step of administering the composition comprising a CD38 agonist. In some of the embodiments where the anti-tumor antibody therapy comprises administration of at least one dose of antibody targeting a tumor antigen, the at least one step of determining CD38 expression level on surfaces of the effector cells is a pre-therapy step of determining in that it is performed prior to at least one particular dose of the anti-tumor antibody therapy.

In some embodiments, the method of treating cancer further comprises at least two steps of determining CD38 expression level on surfaces of the effector cells, wherein: at least a first step of determining CD38 expression level on surfaces of the effector cells is the pre-therapy step of determining; and at least a second step of determining CD38 expression level on surfaces of the effector cells is a post-therapy step of determining in that it is performed after the at least one particular dose of the anti-tumor antibody therapy, and further wherein the step of administering is not performed until at least one post-therapy step of determining detects a significant increase in CD38 expression on surfaces of the effector cells relative to that determined in the pre-therapy step of determining CD38 expression level on surfaces of the effector cells.

In some of the embodiments where the method of treating cancer further comprises one or more of the steps of determining CD38 expression level on surfaces of the effector cells, the step of determining comprises detecting CD38 protein. In some of the embodiments where the method of treating cancer further comprises one or more of the steps of determining CD38 expression level on surfaces of the effector cells, the step of determining comprises detecting a surrogate marker for CD38 expression on surfaces of the effector cells.

In some embodiments, the method of treating cancer further comprises a second step of administering that comprises administering a second agonist, the second agonist being of a cell surface marker other than CD38 whose expression has increased on surfaces of effector cells that mediate antibody-dependent cellular cytotoxicity (ADCC) when such cells are exposed to tumor cells bound by an anti-tumor antibody. In some particular embodiments, such second step of administering is performed a second period of time after the anti-tumor antibody therapy.

The present invention also provides, in a method of treating cancer with anti-tumor antibody therapy, the improvement that comprises: administering to a patient who has received the anti-tumor antibody therapy for a period of time so that the expression of CD38 is induced on the surface of effector cells that mediate antibody-dependent cellular cytotoxicity (ADCC) when such cells are exposed to tumor cells bound by the anti-tumor antibody, a composition comprising a CD38 agonist that increases ADCC.

The present invention also provides methods of enhancing antibody-dependent cellular cytotoxicity (ADCC) of effector cells in a subject in need thereof, the method comprising administering to the subject a CD38 agonist therapy, wherein the CD38 agonist therapy comprises administration of one or more doses of a CD38 agonist according to a regime correlated with elevated ADCC of the effector cells.

The present invention also provides the use of a CD38 agonist for the preparation of a medicament for enhancing the anti-cancer efficacy of an anti-tumor antibody directed against a tumor-specific antigen, wherein said medicament comprises a CD38 agonist that is administered within a period of time subsequent to the administration of the anti-tumor antibody during which period of time the expression of CD38 is increased on the surface of effector cells that mediate antibody-dependent cellular cytotoxicity (ADCC) upon exposure to the said anti-tumor antibody.

The present invention also provides pharmaceutical compositions comprising a CD38 agonist. In some embodiments, such pharmaceutical composition comprising a CD38 agonist is for use in the treatment of cancer in combination with an antibody directed against a tumor-specific antigen, wherein level of CD38 expression on surfaces of effector cells is monitored before or during treatment with the antibody directed against a tumor-specific antigen and the pharmaceutical composition is administered after an increase of CD38 expression level on surfaces of the effector cells is detected.

The present invention also provides kits for enhancing the anti-cancer effect of an antibody directed against a tumor-specific antigen, which kit comprises a CD38 agonist. In some embodiments, the kit further comprises an antibody directed against a tumor-specific antigen. In some embodiments, the kit further comprises a CD134 agonist or a CD137 agonist.

### BRIEF DESCRIPTION OF THE DRAWINGS

FIG. 1. In vitro induction of CD38 expression on the surface of peripheral blood mononuclear cells (PBMCs) and Natural Killer (NK) cells. (A) Flow cytometry analysis of CD38 expression on the surface of PBMCs and NK cells that were purified from the peripheral blood of healthy donors and subsequently cultured for 24 hours, either in medium alone (graphs labeled “PBMC” and “NK*”) or together with CD20-expressing Raji lymphoblastoid cells in the presence of Rituximab (anti-CD20 antibody, 10 μg/mL; graphs labeled “PBMC Act.” And “Act. NK*”). (B) Flow Cytometry analysis of CD56 and CD38 expression on the surface of purified, fresh PBMCs (PBMC) and NK cells (NK) from healthy donors that were cultured for 24 hours, either in medium alone or together with Raji cells (Raji) in the presence of Rituximab (Rit, 10 μg/mL).

FIG. 2. Flow Cytometry analysis of CD56 and CD38 expression on the surface of NK cells that were purified from the peripheral blood of healthy donors and subsequently cultured for 24 hours, either alone in medium containing IgG control antibody (10 μg/mL), or together with HER2-expressing breast cancer cell line (HER18) in the presence of IgG control antibody (10 μg/mL), Rituximab (10 μg/mL), Trastuzumab (anti-HER2 antibody, 10 μg/mL), or Trastuzumab D265A (a Trastuzumab variant that does not bind Fc_{γ}Rs, 10 μg/mL).

FIG. 3. Flow Cytometry analysis of CD38 expression on the surface of NK cells that were purified from peripheral blood samples from patients following anti-cancer antigen therapy. In a first situation, NK cells were obtained from a single patient suffering from Diffuse Large B-cell Lymphoma (DLBCL, CD20-positive) at the indicated time points between 0 (that is, before administration) and 48 hours (such as 2, 4, 6, 10, 12, 14, 16, 18, 20, 22, 24, 30, 36, and 42 hours) after a regular Rituximab administration (A). Percentages of CD38^{hi} NK cells (defined as NK cells whose CD38 expression was greater than 2 logs above Mean Fluorescence Intensity) were determined by Flow Cytometry and then compared with percentages of CD137-positive⁺or CD134-positive NK cells that are were present in the same samples. Similar determination of CD38^{hi} NK cell levels was performed on peripheral blood samples from patients suffering from distinct cancers and having been treated with an appropriate anti-cancer antigen antibody: an anti-HER2 (Trastuzumab) for breast cancer, an anti-CD20 (Rituximab) for NHL (non-Hodgkin's lymphoma), and an anti-EGFR Cetuximab) for Head-and-Neck Cancer (B). The presented analysis aggregates data on CD38 expression from samples that were obtained (at the indicated time points—i.e., the indicated number of hours after administration of a dose of anti-cancer antigen antibody) from 10 patients per histology, each of whom had the indicated cancer and had been treated with the indicated anti-cancer antigen antibody. Represented are 10 patients for each histology characterizing percent of CD38^{hi}. Noted are mean+1 standard error notable for relative peak at approximately 12 hours.

FIG. 4. (A) Peripheral blood samples were obtained from a patient suffering from HER2-expressing breast cancer, either immediately before or 24-hours after a regular Trastuzumab (an anti-HER2 antibody) administration. The expression of CD38 on the surface of the CD3-negative,⁻CD56-positive NK cells in the blood samples was assessed by Flow Cytometry. (B) Peripheral blood samples were obtained from a patient suffering from squamous head and neck carcinoma (an EGFR-positive cancer), either immediately before or 24-72 hours after a regular Cetuximab (an anti-EGFR antibody) administration. The expression of CD38 on the surface of the CD3-negative,⁻CD56-positive NK cells in the blood samples were assessed by Flow Cytometry.

FIG. 5. Agonistic anti-CD38 antibody enhances NK cell cytotoxicity against tumor cells in vitro. PBMCs were first activated with HER2-expressing breast cancer cell line HER18 in the presence of Trastuzumab (10 μg/mL) for 24 hours. The cytotoxicity of the activated PBMCs was subsequently measured by an in vitro ⁵¹Cr release assay, in which the activated PBMCs were incubated with target (HER18) cells at different effector cell:target cell ratios, in the presence of either media alone (i.e., negative control), or of certain monoclonal antibodies (mAbs), namely: an anti-HER2 antibody (Trastuzumab; labeled “αHER2 mAb”), an agonistic anti-CD38 antibody (labeled “αCD38 (IB4) mAb”), an antagonistic anti-CD38 antibody (labeled “αCD38 (HB7) mAb”), or combinations of these anti-CD38 and anti-HER2 antibodies. An agonistic anti-CD137 antibody was also further combined with the anti-HER2 (Trastuzumab) and agonistic anti-CD38 (IB4) antibodies.

FIG. 6. The effect of agonistic anti-CD38 antibody on inhibition of breast tumor growth in vivo was evaluated in nu/nu mice that were inoculated with 5×10⁶ HER2-expressing breast tumor (BT474M1) cells subcutaneously on the right flank. After tumor inoculation, mice received control Rat IgG antibody, anti-HER2 antibody, or agonistic antibody against either CD38 or OX40 on day 3 (d3), day 10 (d10), and day 17 (d17). On day 4 (d4), day 11 (d11), and day 18 (d18), two groups of the mice that had received anti-HER2 antibody further received agonistic antibody against CD38 or OX40. Mice (10 per group) were monitored for tumor growth.

FIG. 7. The effect of an agonistic anti-CD38 antibody on the inhibition of lymphoma growth in vivo was evaluated in a syngeneic lymphoma BALB/c model that were inoculated subcutaneously with 1×10⁶ CD20-expressing A20 tumor cells. After tumor inoculation, mice received a (negative control) rat IgG antibody, an anti-mouse CD20 monoclonal antibody (aCD20; 18B12; 100 μg/dose), or an agonistic anti-mouse CD38 monoclonal antibody (aCD38; NIMR-5; 150 μg/dose) on day 3 (d3), day 10 (d10), and day 17 (d17) as a monotherapy (A). In three other groups of mice that were similarly inoculated with A20 tumor cells, the aCD20 and aCD38 antibodies were administered in the same amount indicated above but in specific combinations: on the same day (at d3, d10, d17), and on consecutive days with either aCD20 administered first (at d3, d10, d17) and then aCD38 (at d4, d11, d18) or in the opposite order (B). Mice (10 per group) were monitored for tumor growth. The asterisk indicates a p value for statistical significance below 0.001.

FIG. 8. The data presented in FIG. 8 show the effect of an agonistic anti-CD38 antibody as a monotherapy or in combination with an anti-CD20 antibody on the inhibition of lymphoma growth in vivo were consolidated in a single graph together with data obtained in a further group of mice that, after tumor inoculation, received aCD20 at d3, d10, d17) and then an agonistic rat anti-mouse CD137 monoclonal antibody (aCD137; IgG2a clone 2A; 150 μg/dose) at d4, d11, and d18. Mice (10 per group) were monitored for tumor growth. The asterisk indicates a p value for statistical significance of 0.015.

FIG. 9. The data presented in FIG. 9 show the ability of various antibodies and antibody combinations to stimulate NK cells in vitro. The figure illustrates that stimulation of NK cells via FcR is the only signal required to upregulate CD137. Following this continued stimulation via the FcR is no longer needed to allow CD137 stimulation via an agonist to enhance NK cell function as measured by degranulation. Regarding CD38, in contrast, initial stimulation via the Fc_{γ}Rs leading to increased CD38 expression apparently must be continued, in order to provide enhanced function as measured by degranulation when a CD38 agonist is used to provide NK cell stimulation. Details on the specific antibodies that are indicated in the Figure (“aCD20”, “aCD38”, and “aCD137”) are present in the text of Example 3. Statistical relevance of p value are indicated by asterisks.

### DEFNITIONS

Below are provided certain definitions of terms used herein, many or most of which confirm common understanding of those skilled in the art.

Administration: As used herein, the term “administration” refers to the administration of a composition to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, within a specific organ (e.g. intrahepatic), mucosal, nasal, oral, rectal, subcutaneous, sublingual, topical, tracheal (including by intratracheal instillation), transdermal, vaginal and vitreal. In some embodiments, administration may involve intermittent dosing. In some embodiments, administration may involve continuous dosing (e.g., perfusion) for at least a selected period of time. As is known in the art, antibody therapy is commonly administered parenterally (e.g., by intravenous or subcutaneous injection).

Agent: The term “agent” as used herein may refer to a compound or entity of any chemical class including, for example, polypeptides, nucleic acids, saccharides, lipids, small molecules, metals, or combinations thereof. As will be clear from context, in some embodiments, an agent can be or comprise a cell or organism, or a fraction, extract, or component thereof. In some embodiments, an agent is or comprises a natural product in that it is found in and/or is obtained from nature. In some embodiments, an agent is or comprises one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present invention include small molecules, antibodies, antibody fragments, aptamers, nucleic acids (e.g., siRNAs, shRNAs, DNA/RNA hybrids, antisense oligonucleotides, ribozymes), peptides, peptide mimetics, etc. In some embodiments, an agent is or comprises a polymer. In some embodiments, an agent is not a polymer and/or is substantially free of any polymer. In some embodiments, an agent contains at least one polymeric moiety. In some embodiments, an agent lacks or is substantially free of any polymeric moiety.

Agonist: As used herein, the term “agonist” refers to an agent whose presence or level correlates with increase in level and/or activity of another agent (i.e., the agonized agent). In general, an agonist may be or include an agent of any chemical class including, for example, small molecules, polypeptides, nucleic acids, carbohydrates, lipids, metals, and/or any other entity that shows the relevant activating activity. An agonist may be direct (in which case it exerts its influence directly upon its target) or indirect (in which case it exerts its influence by other than binding to its target; e.g., by interacting with a regulator of the target, so that level or activity of the target is altered).

Agonist Therapy: The term “agonist therapy”, as used herein, refers to administration of an agonist that agonizes a particular target of interest to achieve a desired therapeutic effect. In some embodiments, agonist therapy involves administering a single dose of an agonist. In some embodiments, agonist therapy involves administering multiple doses of an agonist. In some embodiments, agonist therapy involves administering an agonist according to a dosing regimen known or expected to achieve the therapeutic effect, for example, because such result has been established to a designated degree of statistical confidence, e.g., through administration to a relevant population.

Antagonist: As used herein, the term “antagonist” refers to an agent whose presence or level correlates with decreased level or activity of another agent (i.e., the antagonized agent, or target. In general, an antagonist may be or include an agent of any chemical class including, for example, small molecules, polypeptides, nucleic acids, carbohydrates, lipids, metals, and/or any other entity that shows the relevant inhibitory activity. An antagonist may be direct (in which case it exerts its influence directly upon its target) or indirect (in which case it exerts its influence by other than binding to its target; e.g., by interacting with a regulator of the target, so that level or activity of the target is altered).

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, embodiments, an antibody utilized in accordance with the present invention is in a format selected from, but not limited to, intact IgG, IgE and IgM, bi- or multi-specific antibodies (e.g., Zybodies®, etc), single chain Fvs, polypeptide-Fc fusions, Fabs, cameloid antibodies, masked antibodies (e.g., Probodies®), Small Modular ImmunoPharmaceuticals (“SMIPs™^{”}), single chain or Tandem diabodies (TandAb®), VHHs, Anticalins®, Nanobodies®, minibodies, BiTE®s, ankyrin repeat proteins or DARPINs®, Avimers®, a DART, a TCR-like antibody, Adnectins®, Affilins®, Trans-bodies®, Affibodies®, a TrimerX®, MicroProteins, Fynomers®, Centyrins®, and a KALBITOR®. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]

Antibody Agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to, human antibodies, primatized antibodies, chimeric antibodies, bi-specific antibodies, humanized antibodies, conjugated antibodies (i.e., antibodies conjugated or fused to other proteins, radiolabels, cytotoxins), Small Modular ImmunoPharmaceuticals (“SMIPs™^{”}), single chain antibodies, cameloid antibodies, and antibody fragments. As used herein, the term “antibody agent” also includes intact monoclonal antibodies, polyclonal antibodies, single domain antibodies (e.g., shark single domain antibodies (e.g., IgNAR or fragments thereof)), multispecific antibodies (e.g. bi-specific antibodies) formed from at least two intact antibodies, and antibody fragments so long as they exhibit the desired biological activity. In some embodiments, the term encompasses stapled peptides. In some embodiments, the term encompasses one or more antibody-like binding peptidomimetics. In some embodiments, the term encompasses one or more antibody-like binding scaffold proteins. In come embodiments, the term encompasses monobodies or adnectins. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antibody-Dependent Cellular Cytotoxicity: As used herein, the term “antibody-dependent cellular cytotoxicity” or “ADCC” refers to a phenomenon in which target cells bound by antibody are killed by immune effector cells. Without wishing to be bound by any particular theory, we observe that ADCC is typically understood to involve Fc receptor (FcR)-bearing effector cells can recognizing and subsequently killing antibody-coated target cells (e.g., cells that express on their surface specific antigens to which an antibody is bound). Effector cells that mediate ADCC can include immune cells, including but not limited to one or more of natural killer (NK) cells, macrophage, neutrophils, eosinophils.

Antigen: The term “antigen”, as used herein, refers to an agent that elicits an immune response; and/or (ii) an agent that binds to a T cell receptor (e.g., when presented by an MHC molecule) or to an antibody. In some embodiments, an antigen elicits a humoral response (e.g., including production of antigen-specific antibodies); in some embodiments, an elicits a cellular response (e.g., involving T-cells whose receptors specifically interact with the antigen). In some embodiments, and antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer [e.g., other than a nucleic acid or amino acid polymer) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen.

Biological Sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Biomarker: The term “biomarker” is used herein, consistent with its use in the art, to refer to a to an entity whose presence, level, or form, correlates with a particular biological event or state of interest, so that it is considered to be a “marker” of that event or state. To give but a few examples, in some embodiments, a biomarker may be or comprises a marker for a particular disease state, or for likelihood that a particular disease, disorder or condition may develop. In some embodiments, a biomarker may be or comprise a marker for a particular disease or therapeutic outcome, or likelihood thereof. Thus, in some embodiments, a biomarker is predictive, in some embodiments, a biomarker is prognostic, in some embodiments, a biomarker is diagnostic, of the relevant biological event or state of interest. A biomarker may be an entity of any chemical class. For example, in some embodiments, a biomarker may be or comprise a nucleic acid, a polypeptide, a lipid, a carbohydrate, a small molecule, an inorganic agent (e.g., a metal or ion), or a combination thereof. In some embodiments, a biomarker is a cell surface marker. In some embodiments, a biomarker is intracellular. In some embodiments, a biomarker is found outside of cells (e.g., is secreted or is otherwise generated or present outside of cells, e.g., in a body fluid such as blood, urine, tears, saliva, cerebrospinal fluid, etc.

Cancer: The terms “cancer”, “malignancy”, “neoplasm”, “tumor”, and “carcinoma”, are used interchangeably herein to refer to cells that exhibit relatively abnormal, uncontrolled, and/or autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. The teachings of the present disclosure may be relevant to any and all cancers. To give but a few, non-limiting examples, in some embodiments, teachings of the present disclosure are applied to one or more cancers such as, for example, hematopoietic cancers including leukemias, lymphomas (Hodgkins and non-Hodgkins), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like.

Combination Therapy. As used herein, the term “combination therapy” refers to those situations in which a subject is simultaneously exposed to two or more therapeutic regimens (e.g., two or more therapeutic agents). In some embodiments, two or more agents may be administered simultaneously; in some embodiments, such agents may be administered sequentially; in some embodiments, such agents are administered in overlapping dosing regimens.

Comparable: As used herein, the term “comparable” refers to two or more agents, entities, situations, sets of conditions, etc., that may not be identical to one another but that are sufficiently similar to permit comparison there between so that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: A “composition” or a “pharmaceutical composition” according to this invention refers to the combination of two or more agents as described herein for co-administration or administration as part of the same regimen. It is not required in all embodiments that the combination of agents result in physical admixture, that is, administration as separate co-agents each of the components of the composition is possible; however many patients or practitioners in the field may find it advantageous to prepare a composition that is an admixture of two or more of the ingredients in a pharmaceutically acceptable carrier, diluent, or excipient, making it possible to administer the component ingredients of the combination at the same time.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Dosage Form: As used herein, the term “dosage form” refers to a physically discrete unit of an active agent (e.g., a therapeutic or diagnostic agent) for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing Regimen: As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount In some embodiments, a dosing regimen is correlated with a desired or beneficial outcome when administered across a relevant population (i.e., is a therapeutic dosing regimen).

Inducible Effector Cell Surface Marker: As used herein, the term “inducible effector cell surface marker” refers to an entity, that typically is or includes at least one polypeptide, expressed on the surface of immune effector cells, including without limitation natural killer (NK) cells, which expression is induced or significantly upregulated during activation of the effector cells. In some embodiments, increased surface expression involves increased localization of the marker on the cell surface (e.g., relative to in the cytoplasm or in secreted form, etc). Alternatively or additionally, in some embodiments, increased surface expression involves increased production of the marker by the cell. In some embodiments, increased surface expression of a particular inducible effector cell surface marker correlates with and/or participates in increased activity by the effector cell (e.g., increased antibody-mediated cellular cytotoxicity [ADCC]). In some embodiments, an inducible effector cell surface marker is selected from a group consisting of a member of the TNFR family, a member of the CD28 family, a cell adhesion molecule, a vascular adhesion molecule, a G protein regulator, an immune cell activating protein, a recruiting chemokine/cytokine, a receptor for a recruiting chemokine/cytokine, an ectoenzyme, a member of the immunoglobulin superfamily, a lysosomal associated membrane protein. Certain exemplary inducible cell surface markers include, without limitation, CD38, CD137, OX40, GITR, CD30, ICOS, etc. In some particular embodiments, the term refers to any of the above-mentioned inducible cell surface markers other than CD38.

Patient: As used herein, the term “patient” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes cancer, or presence of one or more tumors. In some embodiments, the patient is receiving or has received certain therapy to diagnose and/or to treat a disease, disorder, or condition.

Pharmaceutically Acceptable: As used herein, the term “pharmaceutically acceptable” applied to the carrier, diluent, or excipient used to formulate a composition as disclosed herein means that the carrier, diluent, or excipient must be compatible with the other ingredients of the composition and not deleterious to the recipient thereof.

Pharmaceutical Composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Refractory: The term “refractory” as used herein, refers to any subject or condition that does not respond with an expected clinical efficacy following the administration of provided compositions as normally observed by practicing medical personnel.

Solid Tumor. As used herein, the term “solid tumor” refers to an abnormal mass of tissue that usually does not contain cysts or liquid areas. Solid tumors may be benign or malignant. Different types of solid tumors are named for the type of cells that form them. Examples of solid tumors are sarcomas, carcinomas, lymphomas, mesothelioma, neuroblastoma, retinoblastoma, etc.

Surrogate Marker: The term “surrogate marker”, as used herein, refers to an entity whose presence, level, or form, may act as a proxy for presence, level, or form of another entity (e.g., a biomarker) of interest. Typically, a surrogate marker may be easier to detect or analyze (e.g., quantify) than is the entity of interest. To give but one example, in some embodiments, where the entity of interest is a protein, an expressed nucleic acid (e.g., mRNA) encoding the protein may sometimes be utilized as a surrogate marker for the protein (or its level). To give another example, in some embodiments, where the entity of interest is an enzyme, a product of the enzyme's activity may sometimes be utilized as a surrogate marker for the enzyme (or its activity level). To give one more example, in some embodiments, where the entity of interest is a small molecule, a metabolite of the small molecule may sometimes be used as a surrogate marker for the small molecule.

Therapeutically Effective Amount: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, stabilizes one or more characteristics of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. For example, in some embodiments, term “therapeutically effective amount”, refers to an amount which, when administered to an individual in need thereof in the context of inventive therapy, will block, stabilize, attenuate, or reverse a cancer-supportive process occurring in said individual, or will enhance or increase a cancer-suppressive process in said individual. In the context of cancer treatment, a “therapeutically effective amount” is an amount which, when administered to an individual diagnosed with a cancer, will prevent, stabilize, inhibit, or reduce the further development of cancer in the individual. A particularly preferred “therapeutically effective amount” of a composition described herein reverses (in a therapeutic treatment) the development of a malignancy such as a pancreatic carcinoma or helps achieve or prolong remission of a malignancy. A therapeutically effective amount administered to an individual to treat a cancer in that individual may be the same or different from a therapeutically effective amount administered to promote remission or inhibit metastasis. As with most cancer therapies, the therapeutic methods described herein are not to be interpreted as, restricted to, or otherwise limited to a “cure” for cancer; rather the methods of treatment are directed to the use of the described compositions to “treat” a cancer, i.e., to effect a desirable or beneficial change in the health of an individual who has cancer. Such benefits are recognized by skilled healthcare providers in the field of oncology and include, but are not limited to, a stabilization of patient condition, a decrease in tumor size (tumor regression), an improvement in vital functions (e.g., improved function of cancerous tissues or organs), a decrease or inhibition of further metastasis, a decrease in opportunistic infections, an increased survivability, a decrease in pain, improved motor function, improved cognitive function, improved feeling of energy (vitality, decreased malaise), improved feeling of well-being, restoration of normal appetite, restoration of healthy weight gain, and combinations thereof. In addition, regression of a particular tumor in an individual (e.g., as the result of treatments described herein) may also be assessed by taking samples of cancer cells from the site of a tumor such as a pancreatic adenocarcinoma (e.g., over the course of treatment) and testing the cancer cells for the level of metabolic and signaling markers to monitor the status of the cancer cells to verify at the molecular level the regression of the cancer cells to a less malignant phenotype. For example, tumor regression induced by employing the methods of this invention would be indicated by finding a decrease in any of the pro-angiogenic markers discussed above, an increase in anti-angiogenic markers described herein, the normalization (i.e., alteration toward a state found in normal individuals not suffering from cancer) of metabolic pathways, intercellular signaling pathways, or intracellular signaling pathways that exhibit abnormal activity in individuals diagnosed with cancer. Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

Subject: By “subject” is meant a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Treatment: As used herein, the term “treatment” (also “treat” or “treating”) refers to any administration of a substance (e.g., anti-receptor tyrosine kinases antibodies or receptor tyrosine kinase antagonists) that partially or completely alleviates, ameliorates, relives, inhibits, delays onset of, reduces severity of, and/or reduces incidence of one or more symptoms, features, and/or causes of a particular disease, disorder, and/or condition (e.g., cancer). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Variant: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a small molecule may have a characteristic core structural element (e.g., a macrocycle core) and/or one or more characteristic pendent moieties so that a variant of the small molecule is one that shares the core structural element and the characteristic pendent moieties but differs in other pendent moieties and/or in types of bonds present (single vs double, E vs Z, etc) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a variant polypeptide may differ from a reference polypeptide as a result of one or more differences in amino acid sequence and/or one or more differences in chemical moieties (e.g., carbohydrates, lipids, etc) covalently attached to the polypeptide backbone. In some embodiments, a variant polypeptide shows an overall sequence identity with a reference polypeptide that is at least 85%, 86%, 87%, 88%, 89%, 90%, 91%, 92%, 93%, 94%, 95%, 96%, 97%, or 99%. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide. In many embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2% of the residues in the variant are substituted as compared with the parent. In some embodiments, a variant has 10, 9, 8, 7, 6, 5, 4, 3, 2, or 1 substituted residue as compared with a parent. Often, a variant has a very small number (e.g., fewer than 5, 4, 3, 2, or 1) number of substituted functional residues (i.e., residues that participate in a particular biological activity). Furthermore, a variant typically has not more than 5, 4, 3, 2, or 1 additions or deletions, and often has no additions or deletions, as compared with the parent. Moreover, any additions or deletions are typically fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, the parent or reference polypeptide is one found in nature. As will be understood by those of ordinary skill in the art, a plurality of variants of a particular polypeptide of interest may commonly be found in nature, particularly when the polypeptide of interest is an infectious agent polypeptide.

### DETAILED DESCRIPTION OF CERTAIN EMBODIMENTS

The present disclosure describes a variety of technologies involving the surprising and beneficial effects of agonizing CD38, particularly in the treatment of cancer. In this section, certain features and particular embodiments of such technologies are discussed in more detail; this discussion is not intended, and should not be construed, to limit the scope of the appended claims, which define the present invention. Rather, it is provided for purposes of illustration and explanation.

CD38

CD38 (cluster of differentiation 38), which is also known both as a receptor and a cyclic ADP ridose hydrolase, is a glycoprotein found on the surface of many immune system cells, including CD4-positive T cells, CD8-positive T cells, B cells, and natural killer cells. CD38 catalyzes the synthesis and hydrolysis of cyclic ADP-ribose (cADPR) from NAD+ to ADP-ribose, thereby playing a role in regulation of intracellular Ca²⁺ and calcium signaling, as well as in cell adhesion and signal transduction.

CD38 is a marker of immune cell activation, and its expression has been linked to rheumatoid arthritis (see, for example, Fueldner et al, 2012), as well as to certain immune and/or blood cell cancers, including diffuse large B-cell lymphoma (DLBCL), acute lymphoblastic leukemia (ALL), acute myeloid leukemia (AML), follicular lymphoma, mantle cell lymphoma, and multiple myeloma (MM) and chronic lymphocytic leukemia (CLL) for which it has been proposed to be an effective target for anti-tumor antibody therapy (see, for example, Malavasi et al., 2011; Chillemi A et al., 2013). Antagonistic antibodies to CD38 are currently in clinical trials (sponsored by Genmab [using daratumumab, developed under the mark HuMax®-CD38], Sanofi [using SAR650984], and MorphoSys AG [using MOR03087]) for the treatment of multiple myeloma (MM). CD38 has been proposed as a useful target for anti-tumor antibody therapy, both to induce ADCC killing of CD38-positive cancer cells, and to potentially deliver payloads (e.g., cytotoxic moieties) to such CD38-positive cancer cells, though caution has been recommended in order to avoid inducing activation signals on target cells (see Chillemi A, et al., 2013).

One reported effect of CD38 ligation (as occurs upon binding of ligand or antibody) is down-regulation of miR-193b, known to function as a tumor suppressor miRNA in various cancers including, for example, non-small cell lung carcinoma, breast cancer, prostate carcinoma, melanoma, hepatocellular carcinoma, etc (see, for example, Chillemi A, et al., 2013 and references cited therein). CD38 ligation has also been reported to induce proliferation and immunoblast differentiation of immune tumor cells such as CLL (see, for example, Chillemi A, et al., 2013).Surface levels of CD38 vary on different cell types, whether due to different expression levels, different distribution of forms (e.g., internalized, soluble, etc), or other differences. For example, surface levels tend to be high for myeloma cells; lower for CLL and some other cells.

CD38 expression is modulated by retinoic acid and other retinoids; the promoter driving transcription of the CD38 gene is responsive to a retinoic acid response element (RARE). It has been proposed that CD38 expression levels might be increased through administration of retinoids or their derivatives, specifically including tamibarotene (see Chillemi A, et al., 2013). However, those retinoids that have been tested showed only a modest ability to increase surface CD38 when administered to myeloma cells, and no effect at all was observed when administered to CLL.

Prior to the present disclosure, therapeutic strategies that were targeting CD38 on tumor cell surfaces, and were designed either to neutralize its pro-cancer activities or to orchestrate tumor cell killing, have been extensively studied. These therapies therefore utilize agents that are non-agonistic, and often antagonistic, to CD38 (see, e.g., Tamibarotene; Drach J et al., 1994; Congleton J et al., 2011; Stevenson G T, 2006; Flavell D et al. 2001; de Weers M et al., 2011; van der Veer M S et al., 2011; WO2008/035257; WO2008/047242; WO2010/061358; WO2011/154453; WO2012/092616; WO2012/076663; US20120189622; US20130302318).

The present disclosure provides an entirely different approach to targeting CD38 in cancer therapy. Indeed, the present disclosure teaches that, exactly opposite to many teachings in the art, effective antibody therapy can be achieved by agonizing CD38. The present disclosure targets CD38 on immune effector cells, rather than CD38 on tumor cells. As described herein, the present invention provides compositions and methods for agonizing CD38 activity, including in the treatment of various cancers. The present disclosure specifically demonstrates that agonizing CD38 on surfaces of immune effector cells (e.g., NK cells) can increase ADCC by such cells. Still further, the present disclosure demonstrates that CD38 agonist therapy can desirably and effectively be combined with anti-tumor antibody therapy to enhance killing of tumor cells bound by specific anti-tumor antibody agents (e.g., antibodies that specifically bind to tumor-associated antigens on tumor cell surfaces).

The biology of CD38 and its downstream signaling cascades in immune effector cells has been well studied. When activated, CD38 induces a flux of calcium ions and triggers the phosphorylation of a cascade of intracellular substrates, leading to the secretion of cytokines and the proliferation and enhanced function of lymphocytes. Despite the knowledge of the molecular mechanism of CD38 action, and in contrast to the extensive study of CD38 as a therapeutic target on tumor cell surface, prior to the present disclosure, significant effort had not been directed to exploring the possibility of agonizing CD38 on the immune effector cells to treat cancer in vivo.

Several reasons may explain this apparent lack of interest in CD38 as a target of agonist therapy. Mature resting immune cells, including effector cells such as NK cells, tend to express very low levels of CD38. Moreover, the many studies that, as described above, focused on blocking CD38 on tumor cells did not report negative impacts on anti-tumor immune responses. Still further, and perhaps most importantly, the understanding that activating CD38 promotes survival and proliferation of tumor cells strongly suggested that agonizing CD38 would be affirmatively undesirable, at least for the treatment of CD38-positive immune or blood cell cancers.

Thus, the present invention surprisingly identifies cell surface CD38 on immune effector cells (e.g., NK cells) as a promising therapeutic target, and provides methods of treating cancer by agonizing CD38. The present disclosure furthermore observes that CD38 on surfaces of immune effector cells (e.g., NK cells) can be induced upon contact with antibody-coated tumor cells in a FcR-dependent manner. In some embodiments, therefore, the present invention provides cancer therapies that involve administering both anti-tumor antibody therapy and CD38 agonist therapy. In some particular embodiments, the CD38 agonist therapy is administered a period of time after the anti-tumor antibody therapy (e.g., after a delay, for example so that an individual dose, or even every individual dose, of CD38 agonist is administered after a delay period following administration of anti-tumor antibody therapy [e.g., of one or more doses of anti-tumor antibody).

The utility and effectiveness of a serial, staged therapy that involve administering an agonist of an inducible effector cell surface marker (e.g., such as described here, including in some particular embodiments, CD137, OX40, GITR, ICOS, etc.) a period of time after an anti-tumor antibody therapy has been previously disclosed. Prior to present invention, however, there were reasons (as discussed above) to doubt the applicability of such therapeutic strategy to CD38 agonist. In particular, there were concerns that it would be affirmatively undesirable to agonize CD38, at least with regard to CD38-positive immune or blood cancers, because agonist activating CD38 on tumor cells can promote the survival and proliferation of the tumor cells.

In some embodiments, therefore, the present invention provides means for augmenting ADCC-mediated cancer cell killing (and consequently reducing tumor size and/or other cancer effects) by administering sequentially two distinct agents: first an antibody agent (which antibody agent includes an immunoglobulin Fc portion) directed against one or more cancer antigens, and after a period of time, an agonist of CD38. The administration of the anti-cancer antibody agent leads to up-regulation of CD38 on the surface of NK cells in the cancer patients (as determined in biological sample obtained from them) and to activation of such cells that can be exploited for enhancing ADCC function only by the subsequent administration of a CD38 agonist, such as an agonistic anti-CD38 antibody. The sequential administration is not intended as reflecting the effects of the administration of said agents within independent monotherapy regimens, since both a precise order and timing are required for obtaining the desired effects on cancer cells.

This approach is shown to improve the clinical efficacy of antibodies directed against cancer-specific antigens that are already used for treating subjects (e.g., patients) or that are still under development. It may be further combined with a standard of care therapy for a cancer (or, if appropriate, this approach may be applied to a cancer patient who has failed standard of care therapy) with the scope of enhancing and/or prolonging ADCC response against cancer cells and consequently the therapeutic effect of the antibody directed against a cancer antigen.

Inducible Effector Cell Surface Markers

Both adaptive and innate immune cells participate in surveillance and elimination of cells dynamically expressing cancer antigens. In particular, the interaction between the Fc portion of the antibodies bound to the antigen on the surface of cancer cells with the Fc receptor (FcR) on the surface of immune effector cells mediates cytotoxic effects of and/or phagocytosis of cancer cells by such effector cells.

Among the effector cells that can destroy tumor cells are natural killer cells (NK cells), which play a major role by releasing small cytoplasmic granules of proteins called perforin and granzyme that cause the target cancer cells to die by apoptosis. NK-cell-mediated lysis of target cells occurs either through spontaneous cytotoxicity, which is modulated by recognition of self versus non-self cell surface markers, or through ADCC. Particularly potent NK-cell-mediated ADCC responses can be triggered by cancer cells to which anti-tumor antibodies (whether naturally generated or administered as part of anti-tumor antibody therapy, for example as described herein) have bound. In fact, in some cases, NK cell-mediated ADCC triggered by Fc_{γ}R engagement with anti-tumor antibodies bound to tumor cell surfaces is one of the primary mechanisms of effective anti-tumor antibody therapy (Weiner G J, 2007).

One event that happens during the activation of effector cells upon the FcR engagement with anti-tumor antibodies bound to tumor cell surfaces is the increase of the expression of various inducible effector cell surface markers on the surface of the effector cells. Activation of such inducible effector cell surface markers can enhance the effector cell function, such as increasing ADCC activity. Such inducible surface markers are known to those of skill in the art, and include, without limitation, certain members of the TNFR family, certain members of the CD28 family, certain cell adhesion molecules, certain vascular adhesion molecules, certain G protein regulators, certain immune cell activating proteins, certain recruiting chemokine/cytokines, certain receptors for recruiting chemokine/cytokines, certain ectoenzymes, certain members of the immunoglobulin superfamily, certain lysosomal associated membrane proteins, and combinations thereof. In some embodiments, inducible effector cell surface markers are selected from CD38 (discussed above), CD137, OX40, GITR, CD30, ICOS, etc.

Many such costimulatory molecules are members of the tumor necrosis factor receptor family (TNFR). TNFR-related molecules do not have any known enzymatic activity and depend on the recruitment of cytoplasmic proteins for the activation of downstream signaling pathways. Members of this receptor family and their structurally related ligands are important regulators of a wide variety of physiologic processes and play an important role in the regulation of immune responses.

CD137, which may also be referred to as Ly63, ILA or 4-1 BB is a member of the tumor necrosis factor (TNF) receptor family. CD137 is expressed by activated NK cells, T and B lymphocytes and monocytes/macrophages. The gene encodes a 255-amino acid protein with 3 cysteine-rich motifs in the extracellular domain (characteristic of this receptor family), a transmembrane region, and a short N-terminal cytoplasmic portion containing potential phosphorylation sites. Expression in primary cells is strictly activation dependent. The ligand for the receptor is TNFSF9. Human CD137 is reported to bind only to its ligand. Agonists include the native ligand (TNFSF9), aptamers (see McNamara et al., 2008), and antibodies. OX40 (CD134) and its binding partner, OX4OL (CD252), are members of the tumor necrosis factor receptor/tumor necrosis factor superfamily and are expressed on activated T cells as well as on a number of other lymphoid and non-lymphoid cells. OX40 and OX4OL regulate cytokine production from T cells, antigen-presenting cells, natural killer cells, and natural killer T cells, and modulate cytokine receptor signaling.

Glucocorticoid-Induced TNFR-Related (GITR) protein belongs to tumor necrosis factor receptor/tumor necrosis factor superfamily and stimulates both the acquired and innate immunity. It is expressed in several cells and tissues, including T and Natural Killer (NK) cells and is activated by its ligand, GITRL, mainly expressed on antigen presenting cells and endothelial cells. GITR/GITRL system participates in the development of autoimmune/inflammatory responses and potentiates response to infection and tumors by mechanisms including NK-cell co-activation.

The transmembrane receptor CD30 (TNFRSF8) and its ligand CD3OL (CD153, TNFSF8) are members of the tumor necrosis factor (TNF) superfamily and display restricted expression in subpopulations of activated immune cells. CD30 is a type I transmembrane glycoprotein of the TNF receptor superfamily. The ligand for CD30 is CD3OL (CD153). The binding of CD30 to CD3OL mediates pleiotropic effects including cell proliferation, activation, differentiation, and apoptotic cell death.

Inducible costimulator (ICOS) is a member of the CD28 family. ICOS expression, may be readily detectable resting, but it upregulated upon activation. ICOS and ICOS-L appear to be a monogamous pair. ICOS activation enhances effector functions.

CD38 Agonists

The present invention, as described herein, provides therapeutic modalities that involve agonizing CD38 on the surface of the effector cells. Some agonists of CD38 are known in the art. Others can be identified, generated, and/or characterized as described herein.

CD31, also known as platelet endothelial cell adhesion molecule-1, PECAM-1, is a CD38 non-substrate ligand that can start the signaling cascade and recapitulates the biological events observed in vitro using agonistic monoclonal antibodies (Malavasi F et al., 2008; Chillemi A, et al. 2013). In some embodiments, therefore, a CD38 agonist can be or comprise the whole or fragments or other variants of the extracellular domain of CD31.

In some embodiments, a CD38 agonist is or comprises an antibody agent (e.g., an intact antibody) specific for human CD38. Antibodies that recognize the extracellular domain of CD38 (and in particular of human CD38) have been generated using different approaches but those appropriate for enhancing the ADCC and cancer cell killing are those having comparable features the CD38-specific agonistic properties as originally described in the literature.

For example, the mouse anti-human CD38 monoclonal antibody, named IB4, induces a rapid mobilization of calcium ions, the phosphorylation of intracellular proteins, cytokine secretion (in particular Interleukin 6 and Interferon gamma), and proliferation of human T lymphocytes (Funaro A et al., 1990). Since the epitope of IB4 antibody has been mapped in the portion of CD38 corresponding to amino acids 220-241 (Ausiello C et al., 2000), antibodies that are CD38 agonists can be raised in animals and/or selected from libraries of antibodies by using such fragment of CD38 as antigen. Alternatively, other monoclonal anti-CD38 antibodies named as CS/2, clone 90 and NIM-R5 (Santos-Argumedo L et al., 1993; Mayo L et al., 2008; Hara-Yokoyama M et al., 2008) provides similar agonistic activities.

In general, an antibody agent that agonizes CD38 may be or comprise an intact antibody, or another antibody format (e.g., as known in the art and/or described herein), including for example a single chain format or a multi-specific format. In some particular embodiments, such a multi-specific agent binds specifically to CD38 and to an inducible immune effector cell surface marker other than CD38. In some particular embodiments, the multi-specific agent binds specifically to CD38 and to a tumor antigen. In some particular embodiments, the multi-specific agent binds specifically to CD38 and to another antigen, which other antigen is not a tumor antigen (so that CD38 and tumor antigen are not simultaneously targeted). In some embodiments that utilize a multi-specific agent that binds specifically to both CD38 and a tumor antigen, the multi-specific agent is not administered to a subject until a period of time has passed (e.g., sufficient to permit an increase in CD38 expression on surfaces of immune effector cells) after the subject received anti-tumor antibody therapy with an antibody agent that did not also target CD38.

Additionally, as with other antibody agents described and/or utilized herein, an antibody agent that agonizes CD38 may be polyclonal or, preferably, monoclonal and/or may be of non-human origin (e.g., of rodent or camel origin) or, preferably, may be chimeric, humanized or, most preferably, human.

In some other embodiments, a CD38 agonist is or comprises a non-antibody agent. In some embodiments, such a non-antibody-agent CD38 agonist is or comprises a nucleic acid, saccharide, lipid, small molecule, metal, or a combination thereof. In some embodiments, a non-antibody-agent CD38 agonist is an aptamer that specifically binds to CD38.

Agonists of Inducible Effector Cell Surface Markers

In some embodiments of the present invention, in addition to a CD38 agonist, a second agonist of an inducible effector cell surface marker is administered to cancer patient who has previously received antitumor antibody therapy to improve the ADCC-mediated anti-tumor therapeutic effect of the antitumor antibody. Many potentially useful agonists of the inducible effector cell surface markers are known in the art. Others can be identified, generated, and/or characterized as described herein.

The physiological ligand for CD137 (CD137L; also, 4-1BBL, TNFSF9, etc.) is a 50 kDa transmembrane glycoprotein expressed by the professional antigen presenting cells (APCs). The soluble CD137L (sCD137L) released from various APCs is capable of binding and activating the CD137 receptor. CD137L-transfectants were shown to stimulate NK cell activation, proliferation, and cytokine release in vitro.

The physiological ligand for OX40 (CD134), OX4OL(CD252; also, TNFSF4), is a transmembrane receptor containing 183 amino acids that is expressed on the surface of activated APCs as a trimer allowing it to bind to three OX40 molecules. OX40-OX40L interactions exert several effects on conventional CD4 and CD8 T cells, NK cells, and NKT cells, including promoting division, survival, and differentiation, and regulating cytokine production.

The physiological ligand for GITR, GITRL (TNFSF18), is a transmembrane protein constitutively expressed on various types of APCs as well as on regulatory T cells. The activation of GITR by GITRL regulates the activity of both conventional and regulatory T cells.

The physiological ligand for CD30, CD30L (CD153; also, TNFSF8), is a transmembrane glycoprotein with expression restricted to and tightly regulated in immune cells. Activation of CD30 by tecombinant CD30L or CD30L-transfectants enhances the activation, proliferation, and various effector functions of both T and B lymphocytes.

The physiological ligand for ICOS, ICOSL (B7-H2), is a transmembrane protein expressed mainly in APCs. Activation of ICOS by ICOSL plays critical role in a variety of lymphocyte activities, including Th2 cell differentiation, T cell proliferation, T helper cell effector function, B cell differentiation, Ig class switch, etc.

In some embodiments, an agonist of an inducible effector cell surface marker for use in accordance with the present invention is or comprises a physiological ligand of the inducible marker, or a fragment or variant thereof (e.g., containing at least the domain mediating the interaction between the ligand and the marker).

Agonistic antibodies against inducible effector cell surface markers have been shown to exert similar biological functions in immune cells as the physiological ligands. In particular, agonist anti-CD137 mAb (urelumab), agonist anti-OX40 mAbs, and agonist anti-GITR mAb (TRX518) have been extensively studied for their anti-tumor therapeutic effects, and have entered clinical trials.

In general, an antibody agent that agonizes an inducible effector cell surface marker may be or comprise an intact antibody, or another antibody format (e.g., as known in the art and/or described herein), including for example a single chain format or a multi-specific format. In some particular embodiments, an antibody agent that agonizes an inducible effector cell surface marker is provided and/or utilized in a multi-specific (e.g. bi-specific) format that also targets CD38.

Additionally, as with other antibody agents described and/or utilized herein, an antibody agent that agonizes an inducible effector cell surface marker may be polyclonal or, preferably, monoclonal and/or may be of non-human origin (e.g., of rodent or camel origin) or, preferably, may be chimeric, humanized or, most preferably, human.

Tumors

Technologies provided herein are useful in the treatment of any tumor.

In some embodiments, a tumor is a hematologic malignancy, including but not limited to, acute lymphoblastic leukemia, acute myeloid leukemia, chronic lymphocytic leukemia, chronic myelogenous leukemia, hairy cell leukemia, AIDS-related lymphoma, Hodgkin lymphoma, non-Hodgkin lymphoma, Langerhans cell histiocytosis, multiple myeloma, or myeloproliferative neoplasms.

In some embodiments, a tumor is a solid tumor, including but not limited to breast carcinoma, a squamous cell carcinoma, a colon cancer, a head and neck cancer, a lung cancer, a genitourinary cancer, a rectal cancer, a gastric cancer, or an esophageal cancer.

In some particular embodiments, a tumor is selected from a lymphoma, a breast tumor, a colon tumor, and a lung tumor.

In some embodiments, a tumor is characterized by no or low expression of CD38 on the surface of tumor cells. In some embodiments, a tumor is characterized by significant expression of CD38 on the surface of tumor cells; in some such embodiments, tumor cells express CD38 on their surfaces at levels significantly higher than non-tumor cells (e.g., in the same individual or as typically found in a population).

In some particular embodiments, a tumor is an advanced tumor, and/or a refractory tumor. In some embodiments, a tumor is characterized as advanced when cancer patients with such tumor are not candidates for conventional chemotherapy.

Anti-Tumor Antibody Therapy

Prior strategies, described above, for targeting CD38 as part of anti-tumor therapy are representative of a class of anti-tumor antibody therapies that are rapidly becoming the standard of care for treatment of many tumors.

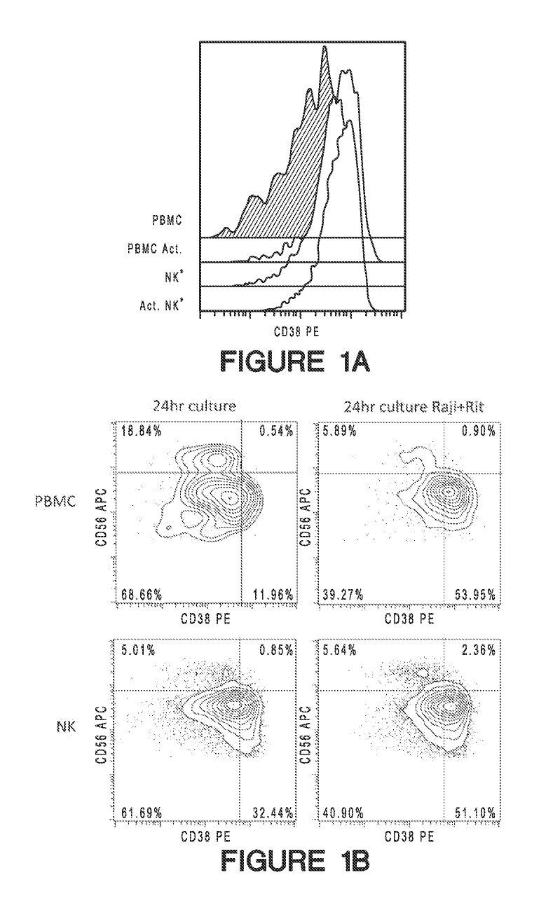

Antibody agents have been designed or selected to bind to tumor cell antigens in order to kill tumor cells by a) delivering a toxic payload associated with the antibody; b) blocking activity of a tumor cell surface receptor that, like CD38, is thought to be involved in cell proliferation and/or survival; c) agonizing activity of a tumor cell surface receptor that is thought to be involved in triggering apoptosis or cell death; and/or d) displaying bound antibody on tumor cell surface, so that immune mechanisms like complement-dependent cytotoxicity (CDC) and/or antibody-dependent cell toxicity (ADCC) are triggered and directed at the tumor (see, for example, review by Scott A M et al., 2012, including particularly FIG. 1 therein). Each of these approaches has been successfully pursued, and several anti-tumor antibody agents are now commercially available for use in cancer therapy.

For example, a steadily increasing number of antibody agents targeting tumor antigens have been approved for use in treating cancers (see, for example, Li G et al., 2013; Scott A M et al., 2012; Sliwkowski M & Mellman I, 2013), and are rapidly becoming standard of care. Indeed, anti-cancer monoclonal antibody therapy can be considered among the most notable scientific advances in the last quarter century for various indications. In particular, the rapid translation of this research towards improved means for targeting molecular targets with a more favorable toxicity profile in comparison with cytotoxic chemotherapy has prolonged the survival of thousands of patients. Monoclonal antibodies against antigens such as CD20, HER2, and EGF receptor have become standard of care for patients suffering from aggressive cancers such as B cell lymphomas, breast cancer, colorectal cancer or head and neck cancers.

Moreover, the list of anti-tumor antibodies in clinical trials seems to expand almost daily (see clinicaltrials.gov). Various review articles have been published that describe useful anti-tumor antibody agents (see, for example, Adler & Dimitrov, 2012; Li G et al., 2013; Scott A M et al., 2012; Sliwkowski M & Mellman I, 2013). The below Table presents a non-comprehensive list of certain human antigens targeted by known, available antibody agents, and notes certain cancer indications for which the antibody agents have been proposed to be useful:

| | Antibody (commercial or | |
|---|---|---|
| Human Antigen | scientific name) | Cancer indication |
| CD2 | Siplizumab | Non-Hodgkin's Lymphoma |
| CD3 | UCHT1 | Peripheral or Cutaneous T-cell |
| CD4 | HuMax-CD4 | Lymphoma |
| CD19 | SAR3419, MEDI-551 | Diffuse Large B-cell Lymphoma |
| CD19 and CD3 or | Bispecific antibodies such as | Non-Hodgkin's Lymphoma |
| CD22 | Blinatumomab, DT2219ARL | |
| CD20 | Rituximab, Veltuzumab, | B cell malignancies (Non-Hodgkin's |
| | Tositumomab, Ofatumumab, | lymphoma, Chronic lymphocytic |
| | Ibritumomab, Obinutuzumab, | leukemia) |
| CD22 (SIGLEC2) | Inotuzumab, tetraxetan, CAT- | Chemotherapy-resistant hairy cell |
| | 8015, DCDT2980S, | leukemia, Hodgkin's lymphoma |
| | Bectumomab | |
| CD30 | Brentuximab vedotin | |
| CD33 | Gemtuzumab ozogamicin | Acute myeloid leukemia |
| | (Mylotarg) | |
| CD37 | TRU-016 | Chronic lymphocytic leukemia |
| CD38 | Daratumumab | Multiple myeloma, hematological |
| | | tumors |
| CD40 | Lucatumumab | Non-Hodgkin's lymphoma |
| CD52 | Alemtuzumab (Campath) | Chronic lymphocytic leukemia |
| CD56 (NCAM1) | Lorvotuzumab | Small Cell Lung Cancer |
| CD66e (CEA) | Labetuzumab | Breast, colon and lung tumors |
| CD70 | SGN-75 | Non-Hodgkin's lymphoma |
| CD74 | Milatuzumab | Non-Hodgkin's lymphoma |
| CD138 (SYND1) | BT062 | Multiple Myeloma |
| CD152 (CTLA-4) | Ipilimumab | Metastatic melanoma |
| CD221 (IGF1R) | AVE1642, IMC-A12, MK-0646, | Glioma, lung, breast, head and neck, |
| | R150, CP 751871 | prostate and thyroid cancer |
| CD254 (RANKL) | Denosumab | Breast and prostate carcinoma |
| CD261 (TRAILR1) | Mapatumumab | Colon, lung and pancreas tumors and |
| CD262 (TRAILR2) | HGS-ETR2, CS-1008 | haematological malignancies |
| CD326 (Epcam) | Edrecolomab, 17-1A, IGN101, | Colon and rectal cancer, malignant |
| | Catumaxomab, | ascites, epithelial tumors (breast, |
| | Adecatumumab | colon, lung) |
| CD309 (VEGFR2) | IM-2C6, CDP791 | Epithelium-derived solid tumors |
| CD319 (SLAMF7) | HuLuc63 | Multiple myeloma |
| CD340 (HER2) | Trastuzumab, Pertuzumab, | Breast cancer |
| | Ado-trastuzumab emtansine | |
| CAIX (CA9) | cG250 | Renal cell carcinoma |
| EGFR (c-erbB) | Cetuximab, Panitumumab, | Solid tumors including glioma, lung, |
| | nimotuzumab and 806 | breast, colon, and head and neck |
| | | tumors |
| EPHA3 (HEK) | KB004, IIIA4 | Lung, kidney and colon tumors, |
| | | melanoma, glioma and haematological |
| | | malignancies |
| Episialin | Epitumomab | Epithelial ovarian tumors |
| FAP | Sibrotuzumab and F19 | Colon, breast, lung, pancreas, and head |
| | | and neck tumors |
| HLA-DR beta | Apolizumab | Chronic lymphocytic leukemia, non- |
| | | Hodkin's lymphoma |
| FOLR-1 | Farletuzumab | Ovarian tumors |
| 5T4 | Anatumomab | Non-small cell lung cancer |
| GD3/GD2 | 3F8, ch14.18, KW-2871 | Neuroectodermal and epithelial tumors |
| gpA33 | huA33 | Colorectal carcinoma |
| GPNMB | Glembatumumab | Breast cancer |
| HER3 (ERBB3) | MM-121 | Breast, colon, lung, ovarian, and |
| | | prostate tumors |
| Integrin αVβ3 | Etaracizumab | Tumor vasculature |
| Integrin α5β1 | Volociximab | Tumor vasculature |
| Lewis-Y antigen | hu3S193, IgN311 | Breast, colon, lung and prostate tumors |
| MET (HGFR) | AMG 102, METMAB, | Breast, ovary and lung tumors |
| | SCH900105 | |
| Mucin-1/CanAg | Pemtumomab, oregovomab, | Breast, colon, lung and ovarian tumors |
| | Cantuzumab | |
| PSMA | ADC, J591 | Prostate Cancer |
| Phosphatidylserine | Bavituximab | Solid tumors |
| TAG-72 | Minretumomab | Breast, colon and lung tumors |
| Tenascin | 81C6 | Glioma, breast and prostate tumours |
| VEGF | Bevacizumab | Tumour vasculature |

Any such anti-tumor antibody agents can be utilized in combination with CD38 agonist therapy in the practice of the present invention. In some embodiments, utilized anti-tumor antibody agents are characterized in that, when effector cells are exposed to tumor cells bound by the anti-tumor antibody agents, CD38 expression on the surface of such effector cells is increased as compared with that observed absent such exposure.

In some embodiments, utilized anti-tumor antibody agents are characterized in that, when effector cells are exposed to tumor cells bound by the anti-tumor antibody agents, the expression of a second inducible effector cell surface marker (e.g., CD137, OX40, GITR, ICOS, CD30, etc.) is also increased on the surface of such effector cells as compared with that observed absent such exposure.

In some embodiments, the appropriate anti-tumor antibody agents are characterized in that, when CD38 agonist is administered to a subject (e.g., a patient) a period of time after the subject receives the anti-tumor antibody agent, ADCC against tumor cells is enhanced as compared with that observed absent the administering of CD38 agonist.

In some embodiments, the appropriate anti-tumor antibody agents are characterized in that, when the subject being administered CD38 agonist therapy has a period of time ago received the anti-tumor antibody agent, ADCC against tumor cells upon the administering of CD38 agonist is increased as compared with that observed absent prior administration of the anti-tumor antibody agent.

In some particular embodiments of the present invention, where a CD20-positive cancer (such as a B cell malignancy) is being treated and anti-tumor antibody therapy is employed, the anti-tumor antibody is specific for CD20, such as Rituximab, Tositumomab, or Ibritumomab.

In some particular embodiments of the present invention, where a CD52-positive cancer (such as leukemia is being treated and anti-tumor antibody therapy is employed, the anti-tumor antibody is specific for CD52, such as Alemtuzumab.

In some particular embodiments of the present invention, where a HER2-positive cancer (such as a solid cancer is being treated and anti-tumor antibody therapy is employed, the anti-tumor antibody is specific for HER2, such as Trastuzumab.

In some particular embodiments of the present invention, where an EGFR-positive cancer (such as a solid cancer is being treated and anti-tumor antibody therapy is employed, the anti-tumor antibody is specific for EGFR, such as Cetuximab;

In some particular embodiments of the present invention, where a CD326-positive cancer (such as a solid cancer) is being treated and anti-tumor antibody therapy is employed, the anti-tumor antibody is specific for CD326, such as Edrecolomab.

In some particular embodiments of the present invention, where a CD38-positive cancer (such as an hematological cancer is being treated and anti-tumor antibody therapy is employed, the anti-tumor antibody is a non-agonist antibody specific for CD38, such as Daratumumab.

It is worth noting that, notwithstanding the promising activity of anti-tumor antibody therapy, and the large number of anti-tumor antibody agents currently under development and/or marketed for treatment of cancer, the response rates among patients are often not night. Particularly with refractory or advanced cancers, response rates can be as low as 25% or less. Efforts to enhance the activity of anti-tumor antibody therapy have often focused on combining the anti-tumor antibody therapy with cytotoxic chemotherapy or radiotherapy (Modjtahedi H et al., 2012). However, these approaches largely ignore and may partially antagonize the immunologic mechanism by which monoclonal antibodies function. The present disclosure, by contrast, demonstrates effective combination of anti-tumor antibody therapy with CD38 agonist therapy. Combination with other agents or modalities, including cytotoxic chemotherapy or radiotherapy, may be included as well, so long as CD38 agonist therapy is utilized.

In some embodiments, appropriate cancer-associated antigens that can desirably be targeted with anti-tumor antibody therapy for use as described herein in combination with CD38 agonist therapy, and/or useful antibody agents that achieve such targeting, will be known to those skilled in the art and/or can be identified and/or characterized via one or more ex vivo, in vivo, or in vitro techniques, as will be familiar to those of skill in the art, reading the present specification.

In some particular embodiments, cancer-associated antigens may be identified and/or characterized by genomic profiling (e.g., that identifies genes for which one or more features of the gene's expression correlates with one or more features of tumor character and/or that identifies genes encoding proteins likely to be partially or wholly displayed on tumor cell surfaces). Alternatively or additionally, in some embodiments, one or more useful cancer-associated antigens may be identified and/or characterized using a technique such as magnetic separation with antibody-coated magnetic beads, “panning” with antibody attached to a solid matrix, mass spectrometry, flow cytometry, proteomic profiling, immunohistochemistry of biopsy samples, and combinations thereof.

Formats of Antibody Agents

A wide variety of formats has been developed for antibody agents, several of which have already progressed into clinical trials (reviewed, for example, in Scott A M et al., 2012). In some embodiments, an antibody utilized in accordance with the present invention is in a format selected from, but not limited to, intact IgG, IgE and IgM, bi- or multi-specific antibodies (e.g., Zybodies®, etc), single chain Fvs, polypeptide-Fc fusions, Fabs, cameloid antibodies, masked antibodies (e.g., Probodies®), Small Modular ImmunoPharmaceuticals (“SMIPs™^{”}), single chain or Tandem diabodies (TandAb®), VHHs, Anticalins®, Nanobodies®, minibodies, BiTE®s, ankyrin repeat proteins or DARPINs®, Avimers®, a DART, a TCR-like antibody, Adnectins®, Affilins®, Trans-bodies®, Affibodies®, a TrimerX®, MicroProteins, Fynomers®, Centyrins®, and a KALBITOR®.

It is worth noting that masked antibody (e.g., Probody®) formats may be of particular interest for certain antibody agents targeting CD38. In some embodiments, use of such a format ensures that CD38 targeting occurs substantially or only in the tumor milieu, and not elsewhere in the body. In some embodiments, use of such a format specifically ensures targeting of CD38 on effector cells in the tumor milieu (e.g., that have infiltrated the tumor).

Combination

Those of ordinary skill in the art, reading the present disclosure, will readily appreciate that CD38 agonistic therapies, as described herein, may in certain embodiments be combined with other anti-cancer therapies, including for example administration of chemotherapeutic agents, other immunomodulatory agents (including other agonists and/or antagonists of other inducible effector cell surface markers), radiation therapy, high-frequency ultrasound therapy, surgery, etc.

Thus, in some embodiments, CD38 agonist therapy, as described herein, is utilized in combination with one or more other therapeutic agents or modalities. In some embodiments, the one or more other therapeutic agents or modalities is also an anti-cancer agent or modality; in some embodiments the combination shows a synergistic effect in treating cancer.

For example, as described herein, in some embodiments, CD38 agonist therapy is combined with anti-tumor anti-body therapy. Alternatively or additionally, in some embodiments, CD38 agonist therapy is combined with agonist therapy targeting an inducible effector cell surface marker other than CD38 and/or with any other compound or treatment known to show therapeutic efficacy in treating cancer.

Known compounds or treatments that show therapeutic efficacy in treating cancer may include, for example, one or more alkylating agents, anti-metabolites, anti-microtubule agents, topoisomerase inhibitors, cytotoxic antibiotics, angiogenesis inhibitors, immunomodulators, vaccines, cell-based therapies (e.g. allogeneic or autologous stem cell transplantation), organ transplantation, radiation therapy, surgery, etc.

Still further, in some embodiments, CD38 agonist therapy (and/or other therapy with which it is combined) may be combined with one or more palliative (e.g., pain relieving, anti-nausea, anti-emesis, etc) therapies, particularly when relieves one or more symptoms known to be associated with the relevant cancer, or with another disease, disorder or condition to which a particular cancer patient is susceptible or from which the particular cancer patient is suffering.

In some embodiments, agents used in combination are administered according to a dosing regimen for which they are approved for individual use. In some embodiments, however, combination with CD38 agonist therapy permits another agent to be administered according to a dosing regimen that involves one or more lower and/or less frequent doses, and/or a reduced number of cycles as compared with that utilized when the agent is administered without CD38 agonist therapy. Alternatively or additionally, in some embodiments, an appropriate dosing regimen involves higher and/or more frequent doses, and/or an increased number of cycles as compared with that utilized when the agent is administered without CD38 agonist therapy.

In some embodiments, one or more doses of agents administered in combination are administered at the same time; in some such embodiments, agents may be administered in the same composition. More commonly, however, agents are administered in different compositions and/or at different times. To give a specific example, as described herein, in many embodiments, agonist therapy that targets an inducible effector cell surface marker (and specifically CD38 agonist therapy) is administered in combination with anti-tumor antibody therapy, and desirably a period of time after administration of (at least one particular dose of) such anti-tumor antibody therapy. As described in detail in the “Dosing and Administration” section herein, in many embodiments, such a period of time separating an administering step of agonist therapy targeting an inducible effector cell surface marker from an administering step of anti-tumor antibody therapy is of sufficient length to permit increased expression of the inducible effector cell surface marker on surfaces of relevant effector cells, desirably so that ADCC (e.g. mediated by the effector cell with elevated surface marker expression) is increased.

Dosing and Administration

Pharmaceutical compositions (e.g., comprising a CD38 agonist, an anti-tumor antibody and/or any other therapeutically active agent) for use in accordance with the present invention may be prepared for storage and/or delivery using any of a variety of techniques and/or technologies known and/or available to those skilled in the art.

In some embodiments, utilized agents (e.g., CD38 agonist, e.g., agonist antibody, anti-tumor antibody, and/or any other therapeutically active agent utilized in accordance with the present invention) is administered according to a dosing regimen approved by a regulatory authority such as the United States Food and Drug Administration (FDA) and/or the European Medicines Agency (EMEA), e.g., for the relevant indication. In some embodiments, however, use of CD38 agonist therapy (e.g., administration of a CD38 agonist) permits reduced dosing (e.g., lower amount of active in one or more doses, smaller number of doses, and/or reduced frequency of doses) of an approved agent used in combination with the CD38 agonist therapy. Those skilled in the art will be aware, or will readily be able to determine, approved dosing regimens for a variety of agents, including for example, a variety of anti-tumor antibodies.

Those skilled in the art, reading the present disclosure will appreciate various modifications of dosing regimens, etc, that are within the scope of the present invention. For example, just to name a few, in some embodiments, CD38 agonist therapy is utilized as monotherapy. In some embodiments, CD38 agonist therapy is combined with other anti-cancer therapies, and particularly with anti-tumor antibody therapy. In some embodiments, one or more doses of CD38 agonist is administered substantially simultaneously with a dose of anti-tumor antibody; in some embodiments, one or more doses of CD38 agonist is administered after a delay relative to a particular dose of anti-tumor antibody; in some embodiments, doses of CD38 agonist are administered after a delay relative to each dose of an anti-tumor antibody. Alternatively or additionally, in some embodiments, CD38 agonist therapy is administered in accordance with the present invention together with lower or less frequent doses of anti-tumor antibody therapy than are standard (e.g., approved) when the anti-tumor antibody therapy is used as monotherapy (or otherwise absent the CD38 agonist therapy). In some such embodiments, addition of yet another anti-cancer therapy may be particularly useful.

Moreover, in some embodiments, it may be desirable to tailor dosing regimens, and particularly to design sequential dosing regimens, based on timing and/or threshold expression levels of inducible markers (including CD38), whether for particular types of tumors, particular tumors, particular patient populations (e.g., carrying genetic markers), and/or particular patients. In some such embodiments, therapeutic dosing regimens may be combined with or adjusted in light of detection methods that assess expression of one or more inducible markers prior to and/or during therapy.

In some embodiments, dosing and administration according to the present invention utilizes active agent having a desired degree of purity combined with one or more physiologically acceptable carriers, excipients or stabilizers in any or variety of forms. These include, for example, liquid, semi-solid and solid dosage forms, such as liquid solutions (e.g., injectable and infusible solutions), dispersions or suspensions, tablets, pills, powders, liposomes and suppositories. In some embodiments, a preferred form may depend on the intended mode of administration and/or therapeutic application. Typical preferred compositions are in the form of injectable or infusible solutions, such as compositions similar to those used for passive immunization of humans with other antibodies.

In some embodiments, ingredient(s) can be prepared with carriers that protect the agent(s) against rapid release and/or degradation, such as a controlled release formulation, including implants, transdermal patches, and microencapsulated delivery systems. Biodegradable, biocompatible polymers can be used, such as polyanhydrides, polyglycolic acid, polyorthoesters, and polylactic acid.

In general, each active agent is formulated, dosed, and administered in therapeutically effective amount using pharmaceutical compositions and dosing regimens that are consistently with good medical practice and appropriate for the relevant agent(s) (e.g., for agents such as antibodies). Pharmaceutical compositions containing active agents can be administered by any appropriate method known in the art, including, without limitation, oral, mucosal, by-inhalation, topical, buccal, nasal, rectal, or parenteral (e.g. intravenous, infusion, intratumoral, intranodal, subcutaneous, intraperitoneal, intramuscular, intradermal, transfermal, or other kinds of administration involving physical breaching of a tissue of a subject and administration of the pharmaceutical composition through the breach in the tissue).

In some embodiments, a dosing regimen for a particular active agent may involve intermittent or continuous (e.g., by perfusion or other slow release system) administration, for example to achieve a particular desired pharmacokinetic profile or other pattern of exposure in one or more tissues or fluids of interest in the subject receiving therapy.

In some embodiments, different agents administered in combination may be administered via different routes of delivery and/or according to different schedules. Alternatively or additionally, in some embodiments, one or more doses of a first active agent is administered substantially simultaneously with, and in some embodiments via a common route and/or as part of a single composition with, one or more other active agents.

Factors to be considered when optimizing routes and/or dosing schedule for a given therapeutic regimen may include, for example, the particular cancer being treated (e.g., type, stage, location, etc), the clinical condition of a subject (e.g., age, overall health, etc), the site of delivery of the agent, the nature of the agent (e.g. an antibody or other protein-based compound), the mode and/or route of administration of the agent, the presence or absence of combination therapy, and other factors known to medical practitioners.

In some embodiments, one or more features of a particular pharmaceutical composition and/or of a utilized dosing regimen may be modified over time (e.g., increasing or decreasing amount of active in any individual dose, increasing or decreasing time intervals between doses, etc), for example in order to optimize a desired therapeutic effect or response (e.g., an ADCC response).

In general, type, amount, and frequency of dosing of active agents in accordance with the present invention in governed by safety and efficacy requirements that apply when relevant agent(s) is/are administered to a mammal, preferably a human. In general, such features of dosing are selected to provide a particular, and typically detectable, therapeutic response as compared with what is observed absent therapy. In context of the present invention, an exemplary desirable therapeutic response may involve, but is not limited to, inhibition of and/or decreased tumor growth, tumor size, metastasis, one or more of the symptoms and side effects that are associated with the tumor, as well as increased apoptosis of cancer cells, therapeutically relevant decrease or increase of one or more cell marker or circulating markers and the like. Such criteria can be readily assessed by any of a variety of immunological, cytological, and other methods that are disclosed in the literature. In particular, the therapeutically effective amount of CD38 agonist, alone or in combination with an third agent, can be determined as being sufficient to enhance ADCC killing of cancer cells targeted by the first agent.

An effective amount of an active agent or composition comprising it can be readily using techniques available in the art including, for example, considering one or more factors such as the disease or condition being treated, the stage of the disease, the age and health and physical condition of the mammal being treated, the severity of the disease, the particular compound being administered, and the like.

In some embodiments, an effective dose (and/or a unit dose) of an active agent, may be at least about 0.01 μg/kg body weight, at least about 0.05 μg/kg body weight; at least about 0.1 μg/kg body weight, at least about 1 μg/kg body weight, at least about 2.5 μg/kg body weight, at least about 5 μg/kg body weight, and not more than about 100 μg/kg body weight. It will be understood by one of skill in the art that in some embodiments such guidelines may be adjusted for the molecular weight of the active agent. The dosage may also be varied for route of administration, the cycle of treatment, or consequently to dose escalation protocol that can be used to determine the maximum tolerated dose and dose limiting toxicity (if any) in connection to the administration of the first agent, second agent, and/or the third agent at increasing doses. Consequently, the relative amounts of the each agent within a pharmaceutical composition may also vary, for example, each composition may comprise between 0.001% and 100% (w/w) of the corresponding agent.

Therapeutic compositions typically should be sterile and stable under the conditions of manufacture and storage. The composition can be formulated as a solution, microemulsion, dispersion, liposome, or other ordered structure suitable to high drug concentration. Sterile injectable solutions can be prepared by incorporating the antibody in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze drying that yields a powder of the active ingredient plus any additional desired ingredient from a previously sterile filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The formulation of each agent should desirably be sterile, as can be accomplished by filtration through sterile filtration membranes, and then packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations as discussed herein. Sterile injectable formulations may be prepared using a non-toxic parenterally acceptable diluent or solvent, such as water or 1,3 butanediol, for example. Other parentally-administrable formulations which are useful include those which comprise the active ingredient in microcrystalline form, in a liposomal preparation, or as a component of biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Each pharmaceutical composition for use in accordance with the present invention may include pharmaceutically acceptable dispersing agents, wetting agents, suspending agents, isotonic agents, coatings, antibacterial and antifungal agents, carriers, excipients, salts, or stabilizers are non-toxic to the subjects at the dosages and concentrations employed. A non-exhaustive list of such additional pharmaceutically acceptable compounds includes buffers such as phosphate, citrate, and other organic acids; antioxidants including ascorbic acid and methionine; salts containing pharmacologically acceptable anions (such as acetate, benzoate, bicarbonate, bisulfate, isothionate, lactate, lactobionate, laurate, malate, maleate, salicylate, stearate, subacetate, succinate, tannate, tartrate, teoclate, tosylate, thiethiodode, and valerate salts); preservatives (such as octadecyidimethylbenzyl ammonium chloride; hexamethonium chloride; benzalkonium chloride, benzethonium chloride; sodium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or antibodies; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN.TM., PLURONICS.TM. or polyethylene glycol (PEG).

In some embodiments, where two or more active agents are utilized in accordance with the present invention, such agents can be administered simultaneousely or sequentially. In some embodiments, administration of one agent is specifically timed relative to administration of another agent. For example, in some embodiments, a first agent is administered so that a particular effect is observed (or expected to be observed, for example based on population studies showing a correlation between a given dosing regimen and the particular effect of interest).

In some embodiments, desired relative dosing regimens for agents administered in combination may be assessed or determined empirically, for example using ex vivo, in vivo and/or in vitro models; in some embodiments, such assessment or empirical determination is made in vivo, in a patient population (e.g., so that a correlation is established), or alternatively in a particular patient of interest.

To give a specific example, in some embodiments of the present invention, CD38 antagonist therapy (and/or other therapy, including specifically agonist therapy, targeting an inducible immune effector cell surface marker) is administered a period of time after administration of anti-tumor antibody therapy. In some such embodiments, the period of time is selected to be correlated with activation of immune effector cells (e.g., NK cells) and/or increased expression of the relevant inducible immune effector cell surface marker (e.g., CD38) on surfaces thereof. In some particular such embodiments, the relevant period of time permits (e.g., is correlated with) increased surface expression of a relevant inducible effector cell surface marker to a level that is at least about 10%, 20%, 50%, 100%, 150%, 200% or more than that observed on the relevant effector cells (e.g., NK cells) prior to (or at the moment of) the administration of the anti-tumor antibody therapy. In some embodiments, surface expression level of the inducible effector cell surface marker is monitored between administration of the anti-tumor antibody therapy and the agonist therapy, for example at one or more specified time points (e.g., for illustration only, at time points such as about 1 hour, 3 hours, 6 hours, 12 hours, and/or 24 hours. In some embodiments, effector cell surface marker expression is monitored in assays that utilize human cancer cells, tissues, and/or other biological materials (such as those obtained from biopsies or blood sample of cancer patients). In some embodiments, the agonist therapy is not administered until a desired level of increased surface expression is achieved. In some embodiments, level of inducible effector cell surface marker is determined via detection of a surrogate marker (e.g., an alternative marker of effector cell (e.g., NK cell) activation, rather than of the inducible effector cell surface marker itself.

In some particular embodiments, administration of agonist therapy targeting an inducible effector cell surface marker (including or specifically CD38) may occur a period of time that starts at least 1 hour, 3 hours, 6 hours, 12 hours, 24 hours, 72 hours, or up to 5 days or more after the administration of anti-tumor antibody therapy. In some embodiments, administration of agonist therapy occurs within a time period during which relevant marker is expressed at an elevated level on surfaces of effector cells (e.g., NK cells). In some embodiments, such a time period (i.e., an “elevated display time period”) begins within an hour or so of administration of anti-tumor antibody therapy and lasts at least about 2, about 5, about 11, about 23 hours, about 71 hours or more. In some embodiments, such a time period lasts between about 1 hour (or less than hour) and about 24 or more hours or about 72 or more hours. In some embodiments, such a time period begins within about 1 hour, about 3 hours, about 6 hours, or about 12 hours of administration of anti-tumor antibody therapy; in some embodiments, such a time period lasts until at least about 12 hours, about 24 hours, about 72 hours, or about 5 days or more after administration of anti-tumor antibody therapy. In some embodiments, such a time period does not last more than about 5 days, about 72 hours, or about 24 hours after administration of anti-tumor antibody.

To give but one illustrative example, a given administration of anti-tumor antibody therapy (e.g., of a given dose of anti-tumor antibody) may achieve a desired increased in inducible effector cell surface for an elevated display time period that lasts at least about 6 hours, and begins about 12 hours after the administration. In such a circumstance, agonist therapy targeting the induced effector cell surface biomarker may desirably be administered according to a regimen (e.g., in a single dose or multiple doses) effective to enhance ADCC against tumor cells against which the anti-tumor antibody therapy was targeted, according to a schedule that achieved administration of the agonist therapy between 12 hours and 18 hours after the administration of the anti-tumor antibody therapy

In some embodiments, one or more active agents utilized in practice of the present invention is administered according to an intermittent dosing regimen comprising at least two cycles. Where two or more agents are administered in combination, and each by such an intermittent, cycling, regimen, individual doses of different agents may be interdigitated with one another. In some embodiments, one or more doses of the second agent is administered a period of time after a dose of the first agent. In some embodiments, each dose of the second agent is administered a period of time after a dose of the first agent. In some embodiments, each dose of the first agent is followed after a period of time by a dose of the second agent. In some embodiments, two or more doses of the first agent are administered between at least one pair of doses of the second agent; in some embodiments, two or more doses of the second agent are administered between al least one pair of doses of the first agent. In some embodiments, different doses of the same agent are separated by a common interval of time; in some embodiments, the interval of time between different doses of the same agent varies. In some embodiments, different doses of the different agents are separated from one another by a common interval of time; in some embodiments, different doses of the different agents are separated from one another by different intervals of time.

To give one exemplary possible protocol for interdigitating two intermittent, cycled dosing regimens (e.g., for anti-tumor antibody therapy and inducible effector cell surface marker therapy), a protocol might include:
a. A first dosing period during which a therapeutically effective amount a first agent is administered to a patient;
b. A first resting period;
c. A second dosing period during which a therapeutically effective amount of a second agent and, optionally, a third agent, is administered to the patient; and
d. A second resting period.

In some embodiments, the first resting period and second resting period may correspond to an identical number of hours or days. Alternatively, in some embodiments, the first resting period and second resting period are different, with either the first resting period being longer than the second one or, preferably, vice versa. In some embodiments, each of the resting periods corresponds to 120 hours, 96 hours, 72 hours, 48 hours, 24 hours, 12 hours, 6 hours, 30 hours, 1 hour, or less. In some embodiments, if the second resting period is longer than the first resting period, it can be defined as a number of days or weeks rather than hours (for instance 1 day, 3 days, 5 days, 1 week, 2, weeks, 4 weeks or more).

If the first resting period's length is determined by existence or development of a particular biological or therapeutic event (e.g., induction of increased surface expression of an inducible effector cell surface marker), then the second resting period's length may be determined on the basis of different factors, separately or in combination. Exemplary such factors may include type and/or stage of a cancer against which anti-tumor antibody therapy (e.g., the first agent) is administered; identity and/or nature of a targeted tumor antigen, identity and/or properties (e.g., pharmacokinetic properties) of the first agent (e.g., of an anti-tumor antibody), and/or one or more features of the patient's response to therapy with the first agent. In some embodiments, length of one or both resting periods may be adjusted in light of pharmacokinetic properties (e.g., as assessed via plasma concentration levels) of one or the other of the administered agents. For example, a relevant resting period might be deemed to be completed with plasma concentration of the relevant agent is below about 1 μg/ml, 0.1 μg/ml, 0.01 μg/ml or 0.001 μg/ml, optionally upon evaluation or other consideration of one or more features of the patient's response (e.g., of degree of cancer reduction and/or magnitude and/or type of induced cancer-specific immune response).

In some embodiments, the number of cycles for which a particular agent is administered may be determined empirically. Also, in some embodiments, the precise regimen followed (e.g., number of doses, spacing of doses (e.g., relative to each other or to another event such as administration of another therapy), amount of doses, etc may be different for one or more cycles as compared with one or more other cycles. Ultimately, patient response is paramount.

Articles of Manufacture and Kits

In another embodiment of the invention, each of first agent, the second agent, and, optionally, the third agent is provided in a separate article of manufacture. In particular, the third agent may target a further antigen on NK cells (such as CD137 and OX40), or further cancer-specific compound selected among chemotherapeutic compounds, cancer vaccines, signal transduction inhibitors, antibodies or other ligands that inhibit tumor growth, and immunomodulatory agents, among many others listed above as a potential third agent.

In another embodiment of the invention, an article of manufacture containing the first agent, the second agent, or, when appropriate, a third agent as described above is provided as a container with a label. Suitable containers include, for example, bottles, vials, syringes, and test tubes. The containers may be formed from a variety of materials such as glass or plastic. The container holds a composition that is effective for treating the condition and may have a sterile access port (for example the container may be an intravenous solution bag or a vial having a stopper pierceable by a hypodermic injection needle). For example, the formulation is packaged in clear glass vials with a rubber stopper and an aluminum seal. The label on, or associated with, the container indicates that the composition is used for treating the condition of choice.

The article of manufacture may further comprise a separate container comprising a pharmaceutically acceptable buffer, such as phosphate-buffered saline, Ringer's solution and dextrose solution. It may further include other materials desirable from a commercial and user standpoint, including other buffers, diluents, filters, needles, syringes, and package inserts with instructions for use. If the second agent and the third agent are simultaneously, the article of manufacture may contain the second agent and the third agent in a single container, or appropriate materials and instructions for reconstituting the second agent and third agent in a single formulation may be provided. For example, the article of manufacture may allow providing each or the agent in an intravenous formulation as a sterile aqueous solution containing a total of 2 mg, 5 mg, 10 mg, 20 mg, 50 mg 100 mg, or more that are formulated, with appropriate diluents and buffers, at a final concentration of 0.1 mg/ml, 1 mg/ml, 10 mg/ml, or at an higher concentration.

Each of the first agent, second agent, and (when applicable) third agent can be provided within the kits-of-parts in the form of lyophilized is to be reconstituted with any appropriate aqueous solution that provided or not with the kits, or other types of dosage unit using any compatible pharmaceutical carrier. As the article of manufacture, this kits-of-parts is labeled for the treatment of a cancer and it may also contain a third agent, as defined above as a further, separate article of manufacture or within the article of manufacture containing the second agent. One or more unit dosage forms of the each of the first agent, the second agent, and, optionally, the third agent may be provided in a pack or dispenser device. Such a pack or device may, for example, comprise metal or plastic foil, such as a blister pack. The kit-of-parts may further comprise materials and/or devices suitable for measuring expression of the target of the first agent, CD38, the target of the third agent, and/or of a surrogate marker, on NK cells (e.g. a detectable labeled reagent that specifically binds to CD38, the target of the third agent, and/or of a surrogate marker, and references for expression). In order to use correctly such kits-of-parts, it may further comprise buffers, diluents, filters, needles, syringes, and package inserts with instructions for use in the treatment of cancer.

The instructions that are associated to the article of manufacture and/or the kits of the invention may be in the form of a label, a leaflet, a publication, a recording, a diagram, or any other means that can be used to inform about the correct use and/or monitoring of the possible effects of the agents, formulations, and other materials in the article of manufacture and/or in the kit. Instructions may be provided together with the article of manufacture and/or in the kit or may be provided separately but with the indication that indications are to be used in association with them.

### EXAMPLES

The invention is further described in detail by reference to the following experimental examples. These examples are provided for purposes of illustration only, and are not intended to be limiting. Thus, the invention should be construed to encompass any and all variations to the following examples which become evident as a result of the teaching provided herein. It is also to be understood that the terminology used herein is for the purpose of describing particular embodiments only, and is not intended to be limiting.

### Example 1

### Agonizing CD38 in Cancer Therapy

The present Example surprisingly demonstrates that agonizing CD38 on immune effector cells can provide effective cancer therapy. In this regard, the present Example demonstrates that level of CD38 on the surface of such immune effector cells can be enhanced by contact with tumor cells to which an anti-cancer antigen antibody is bound. The present Example demonstrates that targeting surface-expressed CD38 on immune effector cells with an agonist antibody can enhance ADCC and decrease tumor load. Moreover, the present Example further surprisingly demonstrates the utility and effectiveness of a serial, staged CD38 agonist therapy relative to anti-tumor antibody therapy.

Prior to the present disclosure, CD38 expressed by tumor cells had been described as a potential target for inhibitory therapy to treat cancer. According to prior understanding, therefore, agonizing CD38 would be affirmatively undesirable for cancer patients.

The present disclosure, however, establishes that administration of a CD38 agonist can augment ADCC capability of immune effector cells (e.g., NK cells). The present disclosure specifically demonstrates that agonizing CD38 can increase effector cell killing of tumor cells.

Moreover, the present Example explicitly establishes that CD38 levels on immune effector cells can be enhanced by contact with tumor cells bound by anti-tumor antibody (i.e., antibody that specifically binds to a tumor antigen). Still further, the present Example demonstrates that administration of a CD38 agonist after such enhancement achieves remarkably effective killing of the tumor cells.

### Materials & Methods

Cell lines and culture. The human breast cancer cell lines BT474M1 (ATCC® HTB-20™) and MCF-7/HER2-18 (MCF-7 cells stably overexpressing HER2, also known as HER18; Benz CC et al.,1992.) were kindly provided as a gift from Byron Hann at UCSF (San Francisco, Calif., USA). The murine CD20-positive cell line, A20, and human CD20-positive B cell line, Raji, was purchased from ATCC. The BT474M1 cell line was cultured in DMEM medium, the Raji cell line in RPMI medium, and the MCF-7/HER2-18 cell line in DMEM/F12 1:1 medium. All media were purchased from Life Technologies. Cells were grown as adherent cultures at 37° C. in 5% CO₂ and passaged after detachment by 0.05% trypsin (Life Technologies). BT474M1 and MCF-7/HER-18 cells express HER2 with specific fluorescence indices (tumor MFI/isotype MFI) of 1.24 and 1.54, respectively. No detectable surface levels of CD38 are observed on these cell lines by flow cytometry assessment.

Mice. Five- to six-week-old female athymic (nu/nu) nude Foxn1^{nu} and SCID mice (Prkdc^{scid}) were purchased from Harlan and Jackson Laboratories and were housed at the Laboratory Animal Facility at the Stanford University Medical Center.

Antibodies. Control rat IgG was purchased from Sigma-Aldrich. Human anti-human agonist CD137 monoclonal antibody (BMS-663513, IgG4) was provided by Bristol-Myers Squibb through a Material Transfer Agreement. Rituximab (murine-human chimeric anti-CD20, IgG1), Trastuzumab (humanized anti-human HER2/neu receptor, IgG1), and Trastuzumab D265A (a variant of Trastuzumab with a single alanine substitution at position 265; Clynes R et al., 2000, Nat Med 6:443) were obtained from Genentech through a Material Transfer Agreement. Agonistic anti-CD134 (OX40) antibody was purchased from BioXcell (clone OX86; Cat. No. BE0031). Mouse anti-human CD38 agonist (IB4) and non-agonistic (HB7) monoclonal antibodies (Funaro A et al., 1990) were kindly donated by Prof. F. Malavasi at Univ. Torino.

Flow cytometry. Monoclonal antibodies against human antigens were used for staining of human PBMCs or purified NK cells, including CD38-PE, CD137-PE, CD56-APC, CD3-PerCP, HER2/neu-APC, CD137-APC, and CD134-APC (all from BD Biosciences). Stained cells were collected on a FACSCalibur or a LSRII 3-laser cytometer (BD Biosciences), and data were analyzed using Cytobank software.

In vitro induction of CD38 expression on human NK cells. PBMCs, which were obtained from the Stanford Blood Center, were isolated from healthy donors by density gradient separation using Ficoll-Paque PLUS (Amersham Biosciences). NK cells were purified by negative magnetic cell sorting using NK cell isolation beads (Miltenyi Biotec). PBMCs and/or purified NK cells were cultured for 24 hours in complete medium alone, in medium containing control Rat IgG alone (10 μg/mL), or with tumor cell line cells (at 1:1 PBMC or NK cell : tumor cell ratio) in the presence of Rituximab, Trastuzumab, or Trastuzumab-D265A (each at 10 μg/mL). Assessment of surface marker expression on NK cells was performed in triplicates for each condition.

CD38 expression on human NK cells from patient samples. PBMCs were obtained from patients suffering from Diffuse Large B-cell Lymphoma (DLBCL, CD20-positive) immediately before and at various time points after Rituximab infusion, and were assayed for NK cell expression of CD137, OX40, and CD38. In some experiments, PBMCs were obtained from patients with HER2/neu-positive breast cancer immediately before and 24 hours after Trastuzumab infusion, and were assayed for CD38 and CD56 expression on CD3⁻CD56⁺ NK cells. In some experiments, PBMCs were obtained from patients with squamous head and neck cancer immediately before and between 24 to 72 hours after Cetuximab infusion, and were assayed for CD38 and CD56 expression on CD3-negative,⁻CD56-positive NK cells.

In vitro NK cell cytotoxicity assays. PBMCs were incubated for 24 hours with irradiated (50 Gy) HER2-expressing breast cancer cells (HER18) at a ratio of 1:1 and with Trastuzumab (10 μg/ml). After 24 hours, NK cells were purified by negative magnetic cell sorting using NK cell isolation beads (Miltenyi Biotec) according to the manufacturer's instructions and to a greater than 90% purity, as defined by CD3-negative and ⁻CD56-positive and confirmed by flow cytometry. Activation of NK cells was confirmed by flow cytometry. NK cell cytotoxicity was additionally measured by a chromium release assay: target cancer cells were labeled with 150 μCi ⁵¹Cr per 1×10⁶ cells for 2 hours, and subsequently added to activated PBMCs at variable effector/target cell ratios from 2:1 to 50:1. Percentage of cell lysis was determined after 4 hours of culture in the presence of the media (i.e., alone), anti-HER2 antibody (Trastuzumab, 10 μg/ml), antagonistic anti-CD38 antibody (HB7, 10 μg/ml), agonistic anti-CD38 antibody (IB4,10 μg/ml), or combinations of this anti-HER2 antibody with an anti-CD38 antibody (each at 10 μg/ml), with or without an agonistic anti-CD137 antibody (10 μg/ml). All assays were performed in triplicate with 3 independent NK cell samples.

Statistics. Prism software (GraphPad) was used to analyze tumor growth and determine statistical significance of differences (including mean±SEM) between groups by applying a 2-tailed, unpaired Student's t test or 2-way ANOVA with Bonferroni's correction for multiple comparisons. P<0.05 was considered significant.

### Results

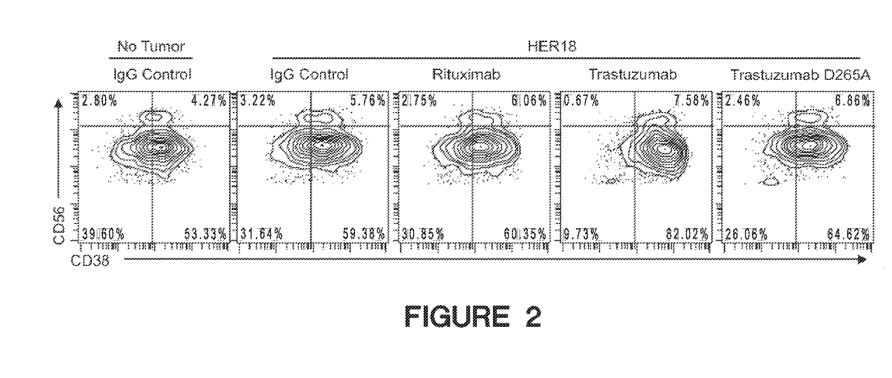

Expression of CD38 on surfaces of PBMCs or NK cells was assessed in an in vitro model. Specifically, preparations of human PBMCs or human NK cells were grown in culture either alone or in the presence of cancer cell lines (either CD20-positive lymphoblastoid B cells or HER2-positive breast cancer cells) that had been exposed to anti-cancer antigen antibody (either Rituximab, which binds to CD20, or Trastuzumab, which binds to HER2). The effect of antibody-coated cancer cells on CD38 expression was measured by flow cytometry. Results are shown in FIG. 1 and FIG. 2.

Specifically, FIGS. 1A and 1B show levels of CD38 expression on PBMCs or NK cells cultured in presence or in absence of Rituximab-treated cancer cells. Flow cytometry analysis was performed either by using a labeled anti-CD38 antibody alone, or this antibody together with a labeled anti-CD56 antibody (CD56 being a non-specific comprehensive marker of NK cell populations). CD38 up-regulation is observed on the surface of purified PBMCs and NK cells from the peripheral blood of healthy donors after being exposed to Rituximab-treated cancer cells (FIG. 1A-B). Thus, the present data demonstrate that CD38 expression level on immune effector cells can be enhanced by contact with tumor cells bound by an anti-cancer antigen antibody.

A similar analysis was performed using a cell line that over-expresses another cancer antigen (HER-2), and is pre-treated either with an antibody specific for such antigen (Trastuzumab) or with control antibodies, including a non-specific IgG, Rituximab (that is specific for CD20, a different cancer antigen), and Trastuzumab D265A (a specifically inactivated variant of Trastuzumab with a single alanine substitution that impairs binding of Trastuzumab to all human FcγR). The increase in cell surface CD38 expression is observed only when the functional variant of the HER-2-specific anti-tumor antibody is used to bind to the HER-2-positive breast cancer cells. The effect on CD38 expression on NK cells following exposure to Trastuzumab-coated HER2-expressing cancer cells is abrogated in the non-FcγR engaging variant, therefore demonstrating that FcγR engagement on NK cells is required for the observed increase in CD38 expression (FIG. 2).

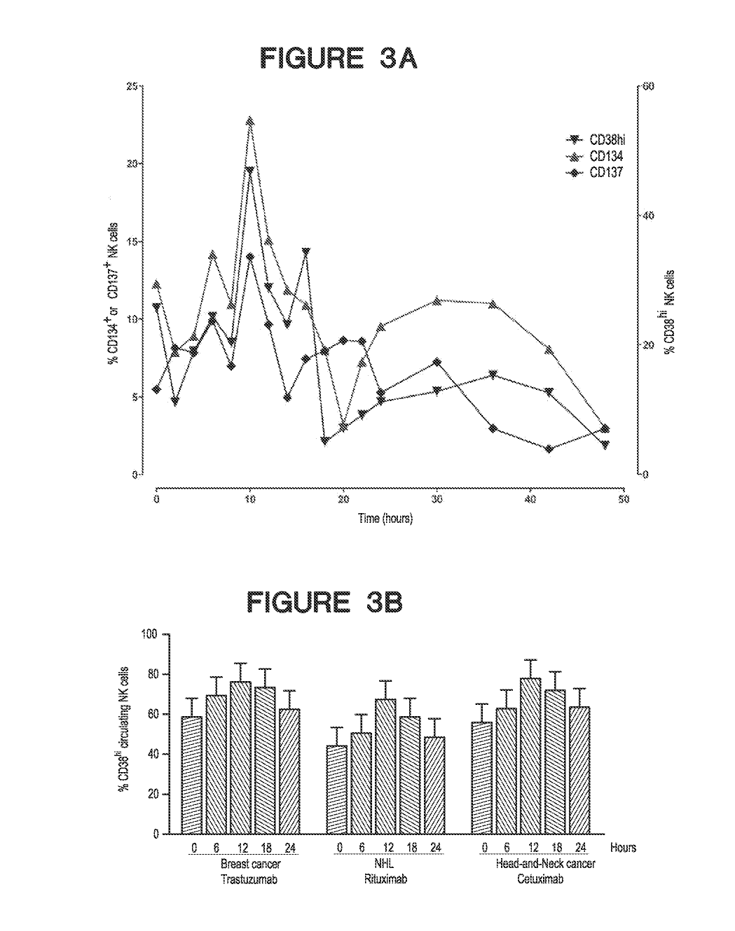

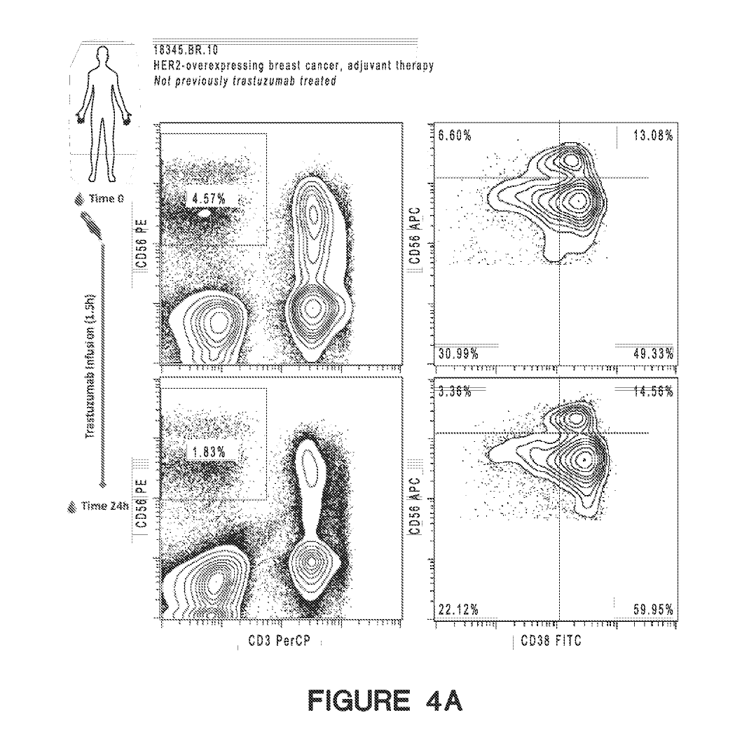

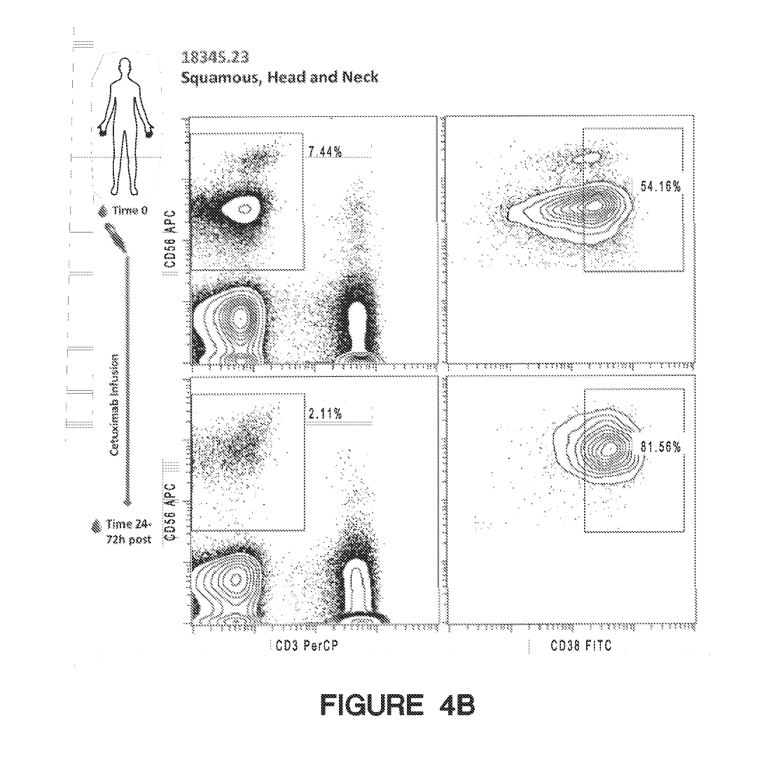

FIG. 3 and FIG. 4 show induction of CD38 and other surface markers on NK cells in vivo. Specifically, they document such induction in blood samples from patients who have been treated with specific anti-tumor antibody therapy.

In particular, FIG. 3A shows CD38 induction in a Non-Hodgkins Lymphoma patient (specifically, a patient suffering from Diffuse Large B-cell Lymphoma; DLBCL) treated with anti-CD20 anti-tumor antibody therapy (Rituximab, which is the standard of care first line therapy, in combination with chemotherapy, for DLBCL). As can be seen, CD38 expression on surfaces of NK cells is induced within a few hours of Rituximab administration, with a peak around 10 hours. FIG. 3A also shows surface expression levels of CD134/OX40 and of CD137 on the same NK cells after the exposure to Rituximab-coated tumor cells, each of which shows a transient increase similar to that observed for CD38.

The analysis presented in FIG. 3A was extended to panels (10 each) of patients with different cancers, each of whom received therapy with anti-cancer antigen antibody appropriate to their tumor. This extended study provided a more general evaluation of the increase of CD38-positive circulating cells as a consequence of different antibody-based therapies, and surprisingly demonstrated a surprisingly similar pattern across tumor types and anti-tumor antibodies. Results of this extended study are presented graphically in FIG. 3B. As can be seen with reference to this FIG. 3B, for all tumors and anti-tumor antibodies tested, CD38 expression had already been detectably increased by the first time point studied, 6 hours after anti-tumor antibody administration. Moreover, in each case, elevated CD38 expression continued to increase and/or was maintained for at least 12, and typically at least 18 hours after anti-tumor antibody administration; in some cases elevated CD38 expression was maintained for at least 24 hours after such administration. Typically, particularly high CD38 expression levels were observed between about 12 hours and up to about 18 hours after administration of anti-tumor antibody. These data reinforce and confirm the findings, presented in FIG. 3A, that were generated through study of a single patient, including specifically the timing of induced CD38 expression on immune effector cells after administration of anti-tumor antibody therapy. In accordance with the present invention, CD38 agonist therapy is most desirably and effectively administered during this period of elevated-CD38 expression.

The findings in FIG. 3 are additionally confirmed by FIG. 4. FIG. 4A shows CD38 induction in breast cancer patients treated with anti-HER2 anti-tumor antibody therapy (Trastuzumab); and FIG. 4B shows CD38 induction in a head and neck cancer patient treated with anti-EGFR anti-tumor antibody therapy (Cetuximab). Specifically, FIGS. 4A and B confirm that the in vivo up-regulation of CD38 expression on NK cells observed for the DLBCL patient in FIG. 3 was also observed in blood samples from patients with HER2-expressing breast cancer or EGFR-expressing squamous head and neck carcinoma and that are treated with Trastuzumab or Cetuximab, respectively. A decrease in percent of circulating NK cells was consistently demonstrated with concurrent up-regulation of CD38 expression on overall CD56-expressing NK cells that are detected in circulation following Trastuzumab or Cetuximab treatment (FIG. 4A-B).

The present invention proposes that antibody-based cytotoxicity against cancer cells may be improved by administering an agonist of CD38 (such as an anti-CD38 agonist antibody) during the time of induced CD38 expression as defined herein. In some embodiments, an anti-CD137 agonist antibody or an anti-CD134/OX40 agonist antibody may be used as a positive control for the timing of CD38 agonist administration (if appropriate).

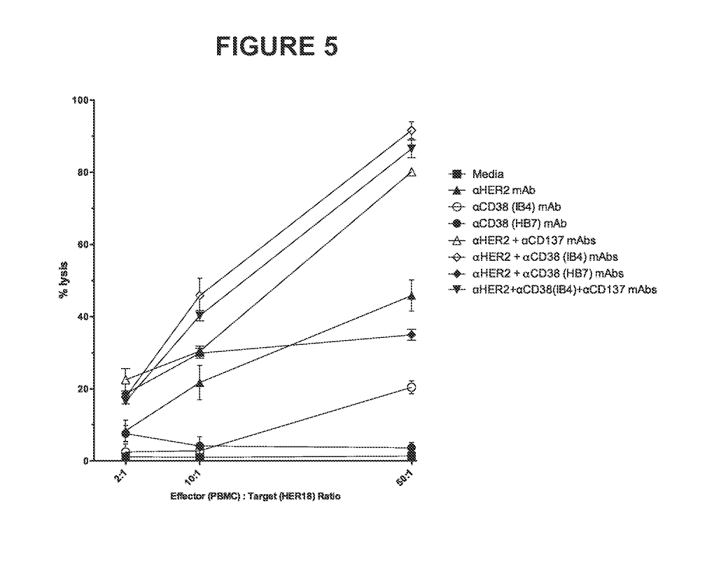

Activated PBMCs (containing CD38-positive NK cells) were incubated with target cells (HER18 breast cancer cells, which express HER2) in the presence of media alone (i.e., negative control), antagonist anti-CD38 antibody (HB7), agonist anti-CD38 antibody (IB4), anti-HER2 antibody (Trastuzumab), combinations of these anti-CD38 and anti-HER2 antibodies, and a combination of anti-HER2 antibody, agonist anti-CD38 antibody, and another agonist antibody targeting a different inducible effector cell surface marker (CD137). FIG. 5 presents the results of these studies.

As can be seen, activated PMBCs alone showed almost no ability to lyse tumor cells. Some cell lysis was observed in the presence of agonistic anti-CD38 antibody (IB4), but not in the presence of antagonistic anti-CD38 antibody (HB7). Significant lysis was observed with anti-tumor antibody (i.e., anti-HER2) alone, but the level was dramatically amplified in the presence of agonistic anti-CD38 antibody (and not in the presence of antagonistic anti-CD38 antibody). Agonistic anti-CD137 antibody also amplified the killing observed in the presence of anti-HER2 antibody, though the effect surprisingly appeared to be less dramatic than that observed with agonistic anti-CD38. In the particular experiment shown in FIG. 5, combined agonistic effects of CD137 or CD38 did not appear to enhance ADCC beyond that observed by agonizing CD38 alone.

### Example 2

### Animal Models and CD38 Agonist Therapy

The present Example confirms the findings described above showing the surprising utility and effectiveness of the administration of a CD38 agonist in cancer therapy, either alone or in combination with anti-tumor antibody therapy. The present Example specifically confirms the surprising utility and effectiveness of a serial, staged combination of anti-tumor antibody therapy and CD38 agonist therapy.

The present Example specifically confirms the findings set forth in Example 1, above, with respect to the utility and effectiveness of CD38 agonist-based therapy in the treatment of at least some cancers. The present Example establishes effectiveness of CD38 agonist therapy as monotherapy, and furthermore confirms the surprising efficacy of combination therapy of a CD38 agonist administered after an anti-tumor antibody.

Still further, the present Example furthermore specifically confirms the relevance and utility of certain animal models for evaluation of CD38 agonist therapies. This finding is particularly noteworthy given that, as will be appreciated by those skilled in the art, interactions between certain immune effector cell inducible markers and ligands that bind thereto are sometimes species-specific, or at least not cross-reactive between human and mouse. For instance, one notable example within the tumor necrosis factor receptor family of inducible immune effector cell surface markers, the GITR/GITRL interaction, is known to be much more species-specific then others such as OX40 (Bossen et al., 2006).

### Materials & Methods

Antibodies. Control rat IgG was purchased from Sigma-Aldrich. Anti-mouse CD20 monoclonal antibody was described (clone 18B12; Ahuja A et al., 1997). Rat anti-mouse CD38 agonist monoclonal antibody (NIMR-5; Harada N et al. 1993) was purchased from Abcam (Cat. No. ab25181). Rat anti-mouse, agonist CD137 monoclonal antibody (IgG2a, clone 2A) was produced from ascites in SCID mice as previously described (Wilcox R et al., 2002).

Transplantation of breast cancer cells and antibody therapy. HER2-positive BT474M1 breast cancer cells were implanted subcutaneously into 5- to 6-week-old female athymic nu/nu mice at a dose of 5×10⁶ cells in 50 μl of PBS mixed with 50 μl of Matrigel (BD Biosciences) 1 day after sub-cutaneous implantation of a 0.72 mg/60 d release β-estradiol pellet (Innovative Research of America). After tumor inoculation, mice received by intraperitoneal (i.p.) injection control Rat IgG antibody (150 μg/injection), Trastuzumab (150 μg/injection), or agonistic antibody to CD38 (NIMR-5, 150 μg/injection) or OX40 (150 μg/injection) on day 3, 10 and 17. On day 4, 11, and 18, two groups of the mice that had received Trastuzumab further received by i.p. injection agonistic antibody against CD38 or OX40. The size of tumor mass was measured by caliper twice a week and expressed as the product of length by width in square centimeters. Mice were sacrificed when tumor size reached 4 cm² or when tumor sites ulcerated. All in vivo models were piloted with 5 mice per group and repeated with 10 mice per group.

Transplantation of lymphoma cells and antibody therapy. CD20-positive, aggressive murine B-cell lymphoma cells (A20; ATCC® TIB-20) were implanted subcutaneously into BALB/c mice (Jackson Lab.) at a dose of 1×10⁶ cells in 50 μl of PBS mixed with 50 μl of Matrigel (BD Biosciences). After tumor inoculation, mice received by intraperitoneal (i.p.) injection control Rat IgG antibody (150 μg/injection), anti-mouse CD20 (100 μg/injection), and/or an agonistic antibody to CD38 (NIMR-5, 150 μg/injection), CD137 (Clone 2A, 150 μg/injection), or OX40 (18B12, 150 μg/injection) on the indicated days and in the indicated combinations. The size of a tumor mass was measured by caliper twice a week and expressed as the product of length by width in square centimeters. Mice were sacrificed when tumor size reached 4 cm² or when tumor sites ulcerated. All in vivo models used 10 mice per group.

Statistics. Prism software (GraphPad) was used to analyze tumor growth and determine statistical significance of differences (including mean±SEM) between groups by applying a 2-tailed, unpaired Student's t test or 2-way ANOVA with Bonferroni's correction for multiple comparisons. P<0.05 was considered significant. For tumor burdens, comparisons of means were done by ANOVA.

### Results

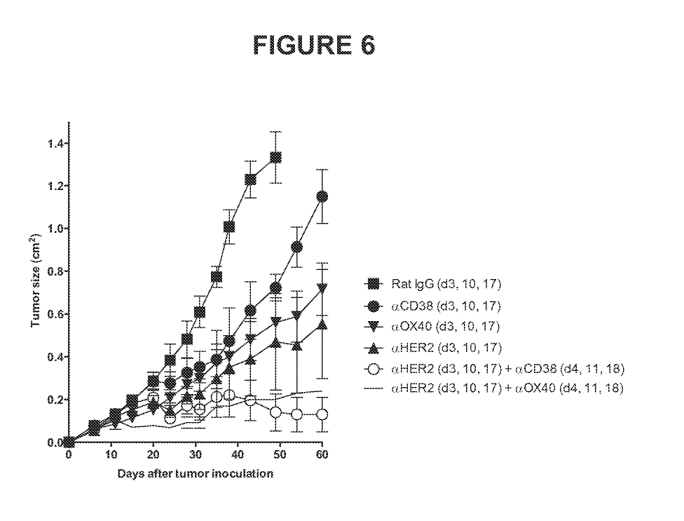

The present Example confirms findings made in Example 1, and particularly confirms that certain animal models can be used to validate and/or evaluate CD38 agonists as valuable tools for cancer therapy. For example, FIG. 6 confirms that the findings depicted in FIG. 5 hold true in an in vivo tumor model. Specifically, a HER2-expressing human cancer cell line was transferred in mice and the growth of tumor was measured in the following weeks during which alternative combinations treatments were tested. Regular administration of either an anti-tumor antibody (anti-HER2) or an agonistic antibody that targets an inducible effector cell surface marker (anti-CD38 or anti-OX40) as monotherapy provided some reduction of growth of the tumor in mice.

Moreover, the therapeutic effect of anti-HER2 antibody (Trastuzumab) administration was dramatically enhanced by the administration of agonist antibody to anti-CD38 or anti-OX40 (FIG. 6). In the particular experiment depicted in FIG. 6, anti-HER2 antibody was dosed three times, and each dose was followed, after a delay (of one day in the particular depicted experiment), by administration of the agonistic antibody. These data confirm, as taught elsewhere herein, that agonizing CD38 can effectively increase the ADCC activity of immune effector cells (e.g. NK cells) that is specifically directed at tumor cells (in particular at breast cancer cells), and that this increase is significantly enhanced when the tumor cells are bound by anti-tumor antibody.

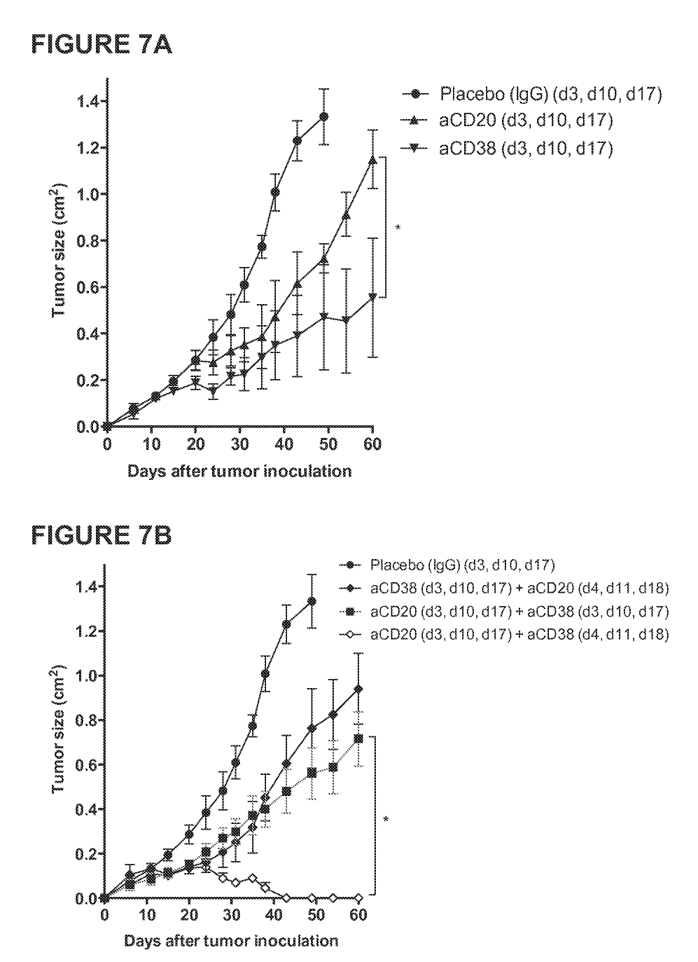

This experimental approach was repeated in a different model wherein lymphoma cells were injected in a syngeneic mouse model, thereby potentially improving understanding of how CD38 agonist-based monotherapy or combination therapies provide therapeutic effects. As shown in FIG. 7A, when compared to placebo (control) antibody treatment, the use of an agonistic anti-CD38 antibodies provides a surprisingly, highly statistically relevant effect in reducing tumor size as a monotherapy when compared with the commonly-used anti-tumor antibody treatment that is directed at CD20.

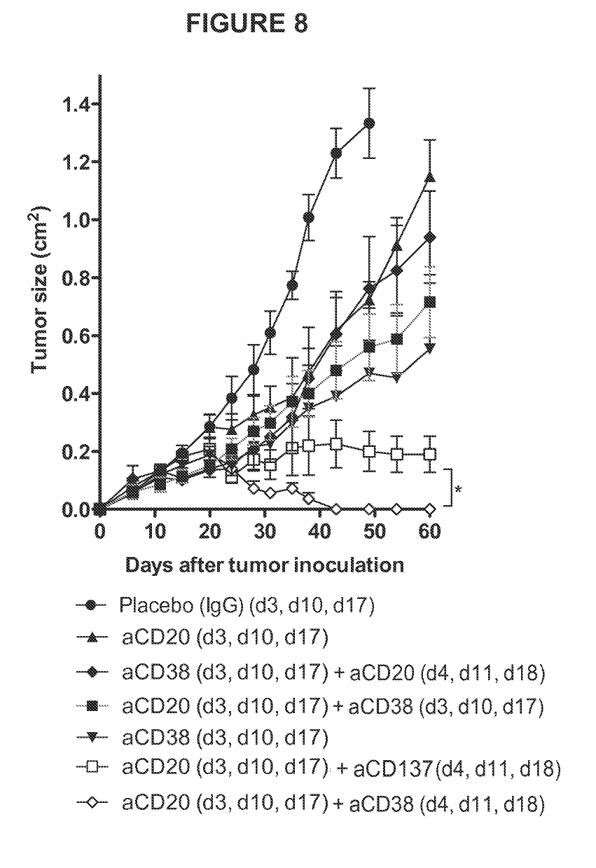

FIG. 7B extends these results by testing the anti-CD20 and agonistic anti-CD38 antibodies in combination with one another, and with different relative timing. The data presented in this FIG. 7B surprisingly demonstrate that demonstrate that, while all combinations effectively inhibit tumor growth, serial administration in which each dose of anti-CD20 antibody is administered a period of time (in this case, one day) prior to a dose of agonist anti-CD38 antibody shows profound synergistic effect, dramatically inhibiting tumor growth far more than any other configuration of dosing (e.g., simultaneous dosing or CD38 agonist dosing prior to anti-CD20 dosing). As is further shown in FIG. 8, administration of an anti-CD20 antibody followed, after a delay, by the administration of an agonistic antibody against CD38 is not only superior to all other tested administration regimens using the anti-CD20 and agonist anti-CD38 antibodies alone or in combination, but is also surprisingly superior to a combination of the anti-CD20 antibody with an agonist antibody against CD137, a different inducible effector cell surface molecule, even when that combination is administered according to the same staggered dosing regimen (i.e., anti-tumor antibody dosing followed, after a delay, by dosing of an agonist antibody against the induced effector cell surface molecule).

### Example 3

### Potential Role of FcR Engagement in CD38 Agonism

The present Example presents data suggesting a role for FcR engagement in effective CD38 agonism. Among other things, the findings presented herein may suggest particularly useful or desirable strategies for timing administration of doses of anti-tumor antibody therapy relative to doses of CD38 agonist therapy.

### Materials & Methods

CD107a mobilization was assayed to evaluate NK-cell degranulation. Purified NK cells isolated by negative magnetic cell sorting using NK cell-isolation beads (Miltenyi Biotec) were cultured alone, with lymphoma cell line (A20) at a ratio of 1:1, with anti-CD20 (18B12)(10 μg/mL), and/or with anti-CD38 mAb (10 μg/mL) or anti-CD137 agonistic mAb (10 μg/mL) for 24 hours with GolgiStop (BD Biosciences) to prevent degradation of reinternalized CD107a proteins. NK cells used for the assay were either fresh, unactivated NK cells (characterized by relatively low CD38 expression, at levels around 40%) or with previously activated NK cells (characterized by high expression of CD38, around 90%) following activation which was achieved by exposure of purified NK cells to 18B12 (10 μg/mL) and A20 tumor cells (at a 1:1 ratio) for 12 hours. After 24 hours, cells were washed 3 times and antibody-based staining was carried out for analysis by flow cytometry. Murine NK cells purified by negative magnetic cell sorting (Miltenyi Biotec) were evaluated for CD38 expression after culture with murine anti-murine anti-CD20 monoclonal antibody.

### Results

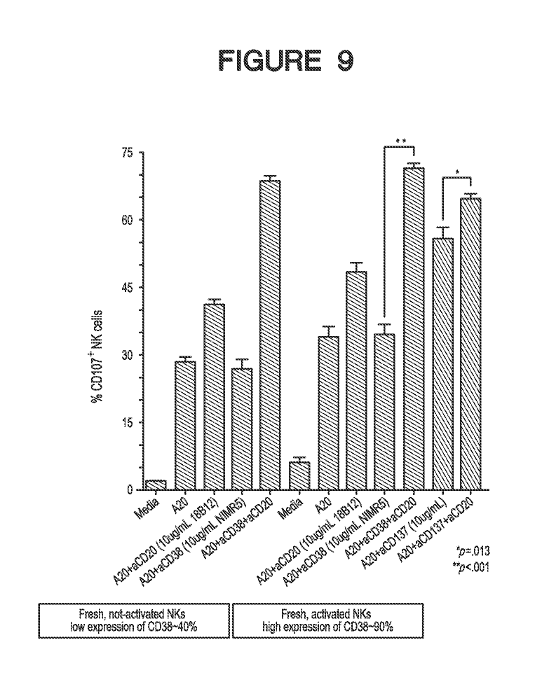

FIG. 9 presents results when A20 lymphoma cells, which express CD20, were contacted with only an anti-CD20 antibody, only an agonist anti-CD38 antibody, or a combination of both antibodies. The left hand side of FIG. 9 shows results obtained when fresh, unactivated NK cells (characterized by relatively low CD38 expression, at levels around 40%) were utilized; the right hand side of FIG. 9 shows results obtained when previously activated NK cells (characterized by high expression of CD38, around 90%) were used. As can be seen, dramatic NK cell activation was observed only when both anti-CD20 and agonist anti-CD38 antibodies were present, even when the NK cells had been previously primed. This finding was wholly unexpected, particularly given that, as shown in the right hand side of FIG. 9, an agonist antibody that bound to a different inducible surface marker on immune effector cells (i.e., to CD137) was almost equally effective when utilized alone or in combination with anti-CD20 antibody therapy to activate already-primed NK cells.

Without wishing to be bound by any particular theory, the present inventors propose that agonism of CD38, which is an ectozyme, may involve one or more different mechanisms than are involved in agonism of certain other inducible effector cell surface markers, such as CD137 specifically. Possibly, one-time priming is insufficient to activate NK cells via CD38 agonism; rather, continued Fc-FcR engagement may be required. If so, then particularly effective dosing regimens may involve administering CD38 agonist therapy while anti-tumor antibody is still present, and preferably engaged on tumor cell surfaces. Certain embodiments of the present invention utilize such regimens, though the invention is not so limited and covers a variety of approaches to agonizing CD38 on immune effector cells, as described herein.

The Examples provided herein explicitly demonstrate that CD38 levels on immune effector cells can be enhanced by contact with tumor cells bound by an anti-tumor antibody (i.e., antibody that specifically binds to a cancer antigen), and further that administration of a CD38 agonist after such enhancement achieves remarkably effective killing of the tumor cells. The present invention therefore provides therapeutic modalities for the treatment of cancer by agonizing CD38, and specifically provides therapeutic modalities for the treatment of cancer by administering a CD38 agonist in combination with anti-tumor antigen therapy. Moreover, the present invention provides technologies increasing the elimination of tumor cells by effector cells by using CD38 agonists. Still further, the present Examples demonstrate effectiveness of such combination when the CD38 agonist is administered after a delay relative to administration of anti-tumor antigen therapy.

Those of ordinary skill in the art, reading the present disclosure, will readily appreciate that CD38 agonistic therapies, as described herein, may in certain embodiments be combined with other anti-cancer therapies, including for example administration of chemotherapeutic agents, other immunomodulatory agents (including other agonists and/or antagonists of other inducible effector cell surface markers), radiation therapy, high-frequency ultrasound therapy, surgery, etc.

Still further, those skilled in the art, reading the present disclosure will appreciate various modifications of dosing regimens, etc, that are within the scope of the present invention. For example, just to name a few, in some embodiments, CD38 agonist therapy is utilized as monotherapy. In some embodiments, CD38 agonist therapy is combined with other anti-cancer therapies, and particularly with anti-tumor antibody therapy. In some embodiments, one or more doses of CD38 agonist is administered substantially simultaneously with a dose of anti-tumor antibody; in some embodiments, one or more doses of CD38 agonist is administered after a delay relative to a particular dose of anti-tumor antibody; in some embodiments, doses of CD38 agonist are administered after a delay relative to each dose of an anti-tumor antibody. Alternatively or additionally, in some embodiments, CD38 agonist therapy is administered in accordance with the present invention together with lower or less frequent doses of anti-tumor antibody therapy than are standard (e.g., approved) when the anti-tumor antibody therapy is used as monotherapy (or otherwise absent the CD38 agonist therapy). In some such embodiments, addition of yet another anti-cancer therapy may be particularly useful.

Thus, it will be appreciated that the demonstrated beneficial effects of CD38 agonism, as documented here, may well also be observed with other CD38 agonists, for example based on other anti-CD38 antibodies (e.g., IB4 or NIM-R5). Useful agonists based on IB4 or NIM-R5 (or other anti-CD38 antibodies or other agonizing agents, including for example small molecule agents), can be identified and/or characterized as described herein. In some embodiments, such agonists are identified and/or characterized by screening recombinant antibodies or natural libraries of antibodies, such as those identified as CD38-specific agonistic autoantibodies in samples from diabetic patients (Antonelli A et al., 2011) and/or by mapping their CD38 binding features in correlation with calcium mobilization and cytokine release, especially associated with the amino acid 220-241 of human CD38 (Ausiello C et al., 2000).

The CD38 agonism-based therapeutic approach described herein may be applied in any of a variety of contexts, including with some modification. To give but a few examples, CD38 agonism can be applied to other CD20-positive lymphomas (for example together with Rituximab or Obinutuzumab), HER2-positive breast cancers (for example together with Trastuzumab as such or conjugated to a cytotoxic drug) or EGFR-positive colorectal or head and neck cancers (for example together with Cetuximab). Alternatively or additionally, a particular agonistic anti-CD134/OX40 or anti-CD137 antibody may be selected for combination with CD38 agonist therapy, including for example through use of multi-specific antibody formats.

Alternatively or additionally, the extensive analysis of the effect of antibodies against cancer antigens on CD38 and CD134/OX40 (or CD137) up-regulation in NK cells from patients or in animal models may provide additional elements for consideration in designing the desirable therapeutic regimens (e.g., sequential regimens), for example, in terms of numbers of doses for each antibody, amount of active in each dose, number of cycles, use of additional therapeutic modalities, target patient populations (e.g., with specific immunological, genetic, and/or cancer marker profiles). In some particular embodiments, an anti-CD134/OX40 and/or anti-CD137 agonist therapy may be utilized in combination with CD38 agonist therapy, including for example through use of multi-specific antibody formats or of formulations comrpising a CD38 agonist and an agonistic anti-CD134/OX40 (or anti-CD137) antibody.

### EQUIVALENTS AND SCOPE

Those skilled in the art will appreciate that the present invention is defined by the appended claims and not by the Examples or other description of certain embodiments included herein. For example, Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, unless specifically excluded limit in the stated range. Where the stated range includes one or both limits, ranges excluding either or both of those limits are also included in the invention.

Similarly, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Unless defined otherwise above, all technical and scientific terms used herein have the same meaning as commonly understood by one of ordinary skill in the art to which this invention belongs. Any methods and materials similar or equivalent to those described herein can also be used in the practice or testing of the present invention. Generally, nomenclatures used in connection with, and techniques of, cell and tissue culture, molecular biology, immunology, genetics and protein and nucleic acid chemistry described herein are those well-known and commonly used in the art, or according to manufacturer's specifications.

Also, all publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### REFERENCES

Adler M J & Dimitrov D S, 2012, Hematol Oncol Clin North Am 26:447.

Ahuja A et al., 1997. J Immunol. 179:3351-6

Antonelli A et al., 2011, Diabetes 5:985.

Ausiello C et al., 2000, Tissue Antigens 56:539.

Benz C C et al.,1992. Breast Cancer Res Treat. 24:85.

Bossen et al., 2006, J Biol Chem 19:13964.

Chillemi A et al., 2013, Mol Med 19:99-108.

Clynes R et al., 2000, Nat Med 6:443

Congleton J et al., 2011, Exp Cell Res 317(7):910.

de Weers M et al., 2011, J Immunol 186:1840.

Drach J et al., 1994, Cancer Res 54:1746.

Flavell D et al., 2001, Br J Cancer 84(4):571.

Fueldner et al., 2012, Arthritis Res Ther 14:R8.

Funaro A et al., 1990, J Immunol 145:2390.

Hara-Yokoyama M et al., 2008, Int Immunopharmacol 8:59.

Harada N et al., 1993, J Immunol. 151: 3111-8 \

Li G et al., 2013, Drug Discov Ther 7:178.

Malavasi F et al., 2008, Physio Rev 88:841.

Malavasi F et al., 2011, Blood 118:3470.

Mayo L et al., 2008, J Immunol 181:92.

McNamara et al., 2008, J Clin Invest 118:376.

Modjtahedi H et al., 2012, Br Med Bull 104:41.

Santos-Argumedo L et al., 1993, J Immunol 151:3119.

Scott A M et al., 2012, Nat Rev Cancer 12:278.

Sliwkowski M & Mellman I, 2013, Science 341:1192.

Stevenson G T, 2006, Mol Med 12:345.

van der Veer M S et al., 2011, Haematologica 96:284.

Weiner G J, 2007, Immunol Res 39:271.

Wilcox R et al., 2002, J Immunol 168:4262

### PATENT REFERENCES

WO2008/035257

WO2008/047242

WO2010/061358

WO2011/154453

WO2012/076663

WO2012/092616

US20120189622

US20130302318

1. A method of treating cancer in a patient who has received anti-tumor antibody therapy, the method comprising:
administering to the patient a composition comprising a CD38 agonist, the administering being performed a period of time after the anti-tumor antibody therapy, such that CD38 expression has increased on surfaces of effector cells that mediate antibody-dependent cellular cytotoxicity (ADCC) when such cells are exposed to tumor cells bound by an anti-tumor antibody;
the CD38 agonist being characterized in that, when the effector cells with increased CD38 expression on their surface are contacted with the agonist, their ADCC is increased as compared with that observed absent such contact; and
wherein the CD38 agonist is or comprises an agonistic anti-human CD38 antibody.

2. The method of claim 1, further comprising at least one step of:
determining CD38 expression level on surfaces of the effector cells, the determining being performed prior to the step of administering the composition comprising a CD38 agonist.

3. The method of claim 2, wherein the anti-tumor antibody therapy comprises administration of at least one dose of antibody targeting a tumor antigen and the at least one step of determining CD38 expression level on surfaces of the effector cells is a pre-therapy step of determining in that it is performed prior to at least one particular dose of the anti-tumor antibody therapy.

4. (canceled)

5. The method of claim 3, wherein the step of determining CD38 expression level on surfaces of the effector cells comprises:
providing a patient sample; and
determining the level in the sample wherein the patient sample is a patient blood sample or cellular fraction thereof.

6-8. (canceled)

9. The method of claim 1, wherein the period of time has a length between 1 hour and 5 days.

10. The method of claim 1, further comprising a second step of administering that comprises administering a second agonist, the second agonist being of a cell surface marker other than CD38 whose expression has increased on surfaces of effector cells that mediate antibody-dependent cellular cytotoxicity (ADCC) when such cells are exposed to tumor cells bound by an anti-tumor antibody wherein the second step of administering is performed a second period of time after the anti-tumor antibody therapy.

11. (canceled)

12. The method of claim 10, wherein the second agonist is or comprises a CD134 agonist or a CD137 agonist.

13. (canceled)

14. The method of claim 1, wherein the effector cells that mediate ADCC are CD3-negative and CD56-positive NK cells.

15. The method of claim 1, wherein the anti-tumor antibody is a monoclonal antibody, a xenogeneic human antibody, a humanized antibody, or a chimeric antibody.

16-33. (canceled)

34. A pharmaceutical composition comprising a CD38 agonist, wherein said composition is for use in the treatment of cancer in combination with an antibody directed against a tumor-specific antigen; and wherein the CD38 agonist is or comprises an agonistic anti-human CD38 antibody.

35-49. (canceled)

50. The method of claim 1, wherein the CD38 agonist is or a humanized or a chimeric anti-human CD38 antibody.

51. The method of claim 1, wherein the antibody directed against a tumor-specific antigen is directed against a specific cancer epitope, or combination of epitopes, that allows the targeting or depletion of cancer cell populations expressing said antigen.

52. The method of claim 1, wherein the cancer is a B cell malignancy.

53. The method of claim 1, wherein the cancer is marginal zone lymphoma, Hodgkins Lymphoma, non-Hodgkins lymphoma, chronic lymphocytic, leukemia, acute lymphoblastic leukemia, myelomas, or myeloproliferative disorders.

54. The method of claim 1, wherein the cancer is a CD20-positive tumor and the antibody directed against a tumor antigen is specific for CD20.

55. The method of claim 1, wherein the cancer is a solid tumor.

56. The method of claim 1, wherein the cancer is a breast carcinoma, a squamous cell carcinoma, a colon cancer, a head and neck cancer, a lung cancer, a genitourinary cancer, a rectal cancer, a gastric cancer, or an esophageal cancer.

57. The method of claim 1, wherein the cancer is a HER2-positive tumor and the antibody selective for a cancer cell antigen is specific for HER2.

58. The method of claim 1, wherein the cancer is a CD20-positive tumor and the antibody directed against a tumor antigen is Rituximab, Tositumomab, or Ibritumomab.

59. The method of claim 1, wherein the cancer is a solid tumor and the antibody directed against a tumor antigen is Trastuzumab or Cetuximab.
